(19) United States
(12) Patent Application Publication
(10) Pub. No.: US 2026/0088139 A1
(43) Pub. Date: Mar. 26, 2026

(54) METHOD FOR LEARNING 3D GEOMETRY OF MOLECULE AND TARGET PHYSICAL PROPERTY PREDICTION METHOD INCLUDING SAME

(71) Applicant: LG MANAGEMENT DEVELOPMENT INSTITUTE CO., LTD., Seoul (KR)

(72) Inventors: Sung Jun CHO, Seoul (KR); Dae Woong Jeong, Seoul (KR); Sung Moon Ko, Gimpo-si (KR); Se Hui Han, Seoul (KR); Hong Lak Lee, Seoul (KR); Moon Tae Lee, Goyang-si (KR)

(21) Appl. No.: 19/407,850

(22) Filed: Dec. 3, 2025

Related U.S. Application Data
(63) Continuation of application No. PCT/KR2024/013648, filed on Sep. 9, 2024.

(30) Foreign Application Priority Data
Mar. 22, 2024 (KR) 10-2024-0039992
Sep. 9, 2024 (KR) 10-2024-0122591

(51) Int. Cl. G16C 20/70; G06N 3/045; G06N 3/096; G16C 20/30

(52) U.S. Cl. CPC G16C 20/70; G06N 3/045; G06N 3/096; G16C 20/30

(57) ABSTRACT

A method for learning a 3D geometric structure of a molecule and a target property prediction method including the same concern a target property prediction method in which a computing system including a memory and a processor learns a 3D geometric structure of a molecule and predicts a target property. The method includes: performing denoising-based, first pre-training based on a 3D conformer encoder which takes, as input, 3D molecular data specifying a 3D-level molecular structure; performing distillation-based, second pre-training based on the first pre-trained 3D conformer encoder and a 2D graph encoder which takes, as input, 2D molecular data specifying a 2D-level molecular structure; performing fine-tuning-based third pre-training based on the second pre-trained 2D graph encoder; and providing the third pre-trained 2D graph encoder.

### CROSS REFERENCE TO RELATED APPLICATIONS

This application is a Bypass Continuation of International Patent Application No. PCT/KR2024/013648, filed on Sep. 9, 2024, which claims priority from and the benefit of Korean Patent Application No. 10-2024-0039992, filed on Mar. 22, 2024 and Korean Patent Application No. 10-2024-0122591, filed on Sep. 9, 2024, each of which is hereby incorporated by reference for all purposes as if fully set forth herein.

### BACKGROUND

### Field

Embodiments of the invention relate generally to a method for learning a 3D geometric structure of a molecule and a target property prediction method including the same, and more particularly, to a method for learning a 3D geometric structure of a molecule, in which a two-dimensional data-based encoder is pre-trained based on a denoise and distill (D&D) methodology using three-dimensional data, and a target property prediction method including the same.

### Discussion of the Background

Prediction of molecular properties plays a crucial role in various fields of chemistry and life science such as drug development and/or novel material design. However, the cost to obtain accurate labeled data required for high-precision prediction is generally high. Therefore, in many cases, it is essential to learn effective molecular representations from large-scale unlabeled molecular data.

Traditionally, two-dimensional graph-based molecular pre-training techniques have been widely used. These methods represent a molecule as a graph composed of atoms and bonds, and utilize graph neural networks to predict molecular properties. However, such 2D graph-based approaches can suffer from damage to the topology of the graph in a data augmentation process, which poses limitations in achieving a meaningful improvement in prediction performance.

For this reason, pre-training methods using three-dimensional (3D) conformers have recently been gaining attention. 3D conformers provide positional information of atoms in a physical space, and can learn the chemical properties of a molecule more accurately using such information. In particular, learning a force field or the like generated from a stabilization process through a 3D structural denoising operation is considered to be highly effective.

However, 3D conformer-based learning methods often require accurate 3D structural information for downstream tasks, making them computationally expensive to apply to actual large-scale data. For instance, generating a 3D conformer for a new molecule involves costly quantum mechanical calculations.

Therefore, to overcome the aforementioned limitations, there is a need for efficient prediction models that do not require 3D information.

The above information disclosed in this Background section is only for understanding of the background of the inventive concepts, and, therefore, it may contain information that does not constitute prior art.

### SUMMARY

Embodiments of the invention have been devised to address the above-described problems of the related art, and is capable of providing a method for learning a 3D geometric structure of a molecule, in which a two-dimensional data-based encoder is pre-trained based on a denoise and distill (D&D) framework using three-dimensional data, and a target property prediction method including the same.

Specifically, embodiments of the invention are capable of providing a method for learning a 3D geometric structure of a molecule, in which denoising-based learning is performed through a three-dimensional conformer encoder, knowledge learned by the three-dimensional conformer encoder is transferred (distilled) to a two-dimensional graph encoder, and the knowledge-distilled, two-dimensional graph encoder is fine-tuned to a particular property, and a target property prediction method including the same.

Additional features of the inventive concepts will be set forth in the description which follows, and in part will be apparent from the description, or may be learned by practice of the inventive concepts.

According to one or more embodiments of the invention, a method for learning a 3D geometric structure of a molecule and a target property prediction method including the same, concern a target property prediction method in which a computing system including a memory and a processor learns a 3D geometric structure of a molecule and predicts a target property, the computer-implemented method includes: performing denoising-based, first pre-training based on a 3D conformer encoder configured to input 3D molecular data specifying a 3D-level molecular structure; performing knowledge distillation-based, second pre-training based on the first pre-trained 3D conformer encoder and a 2D graph encoder configured to input 2D molecular data specifying a 2D-level molecular structure; performing fine-tuning based, third pre-training based on the second pre-trained 2D graph encoder; and providing the third pre-trained 2D graph encoder.

The performing of first pre-training may include: inserting a predetermined noise into the 3D molecular data; and training the 3D conformer encoder to restore the 3D molecular data with the inserted noise to the original 3D molecular data and learn the 3D-level molecular structure.

The performing of first pre-training may further include learning data representations invariant to rotations and translations in a 3D space, based on a predetermined SE(3) permutation invariant architecture.

The performing of second pre-training may include performing distillation training in which a 3D conformer denoising encoder, which is the first pre-trained 3D conformer encoder, serves as a teacher model, and the 2D graph encoder serves as a student model.

The performing of distillation training may include training the 2D graph encoder so that representations outputted by the 2D graph encoder follow representations outputted by the 3D conformer denoising encoder.

The performing of distillation training may further include performing graph-level knowledge distillation (D&D-GRAPH) to minimize the differences between graph-level representations outputted by the 2D graph encoder and graph-level representations outputted by the 3D conformer denoising encoder.

The performing of distillation training may further include performing node-level knowledge distillation (D&D-NODE) to minimize the differences between node-level representations outputted by the 2D graph encoder and node-level representations outputted by the 3D conformer denoising encoder.

The performing of distillation training may further include freezing at least some parameters of the 3D conformer denoising encoder.

The performing of third pre-training may further include performing a downstream task to optimize a 2D graph transfer encoder which is the second pre-trained 2D graph encoder.

The providing of the third pre-trained 2D graph encoder may include applying a 2D graph fine-turning encoder, which is the third pre-trained 2D graph encoder, to a predetermined multitasking model.

The providing of the third pre-trained 2D graph encoder may include: evaluating the feasibility of first input information specifying a plurality of target properties inputted by a user, through a multitasking model including the encoder; and providing guidance based on a result of the evaluation. The providing of guidance may include: acquiring a feasibility indicator quantitatively specifying the difficulty of generation of output data from the multitasking model based on the first input information; and if the acquired feasibility indicator is below a preset reference value, generating and outputting guidance information for decreasing the difficulty of generation.

The acquiring of a feasibility indicator may include calculating the feasibility indicator for the first input information based on at least one of a predetermined density estimation algorithm, a predetermined anomaly detection algorithm, or a predetermined similarity assessment algorithm.

The guidance information may include at least one of first guidance information which suggests making changes to the target properties to decrease the difficulty of generation, or second guidance information which suggests supplementing the training data to improve the model's performance.

According to yet another embodiment of the invention, a system for learning a 3D geometric structure of a molecule and predicting a target property includes: at least one memory and at least one processor configured to retrieve at least one application stored in the memory to learn a 3D geometric structure of a molecule and predict a target property. Instructions of the processor include instructions for executing the steps of: performing denoising-based, first pre-training based on a 3D conformer encoder configured to input, 3D molecular data specifying a 3D-level molecular structure; performing knowledge distillation-based, second pre-training based on the first pre-trained 3D conformer encoder and a 2D graph encoder configured to input 2D molecular data specifying a 2D-level molecular structure; performing fine-tuning-based, third pre-training based on the second pre-trained 2D graph encoder; and providing the third pre-trained 2D graph encoder.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the inventive concepts.

FIG. 1 illustrates an example block diagram of a computing system implementing a model encoder training service according to an embodiment of the invention.

FIG. 2 illustrates an example block diagram of a computing device implementing a model encoder training service according to one embodiment of the invention.

FIG. 3 illustrates an example block diagram of another aspect of a computing device implementing a model encoder training service according to one embodiment of the invention.

FIG. 4 and FIG. 5 illustrate example conceptual diagrams for explaining a multitasking learning model according to one embodiment of the invention.

FIG. 6 illustrates an internal block diagram of a multitasking learning model according to an embodiment of the invention.

FIG. 7 illustrates an example conceptual diagram for explaining a multitasking model training method according to an embodiment of the invention.

FIG. 8 illustrates a block flow diagram for explaining a multitasking model training method according to an embodiment of the invention.

FIG. 9 illustrates a block flow diagram for explaining a method of training a multitasking learning model according to an embodiment of the invention.

FIG. 10 illustrates an example conceptual diagram for explaining a method of training a multitasking learning model according to an embodiment of the invention.

FIG. 11 and FIG. 12 illustrate exemplary diagrams for explaining a method of calculating regression loss according to an embodiment of the invention.

FIG. 13 illustrates an exemplary diagram for explaining a method of mapping to a unified latent space according to an embodiment of the invention.

FIG. 14 and FIG. 15 illustrate exemplary diagrams for explaining a method of calculating consistency loss according to an embodiment of the invention.

FIG. 16 and FIG. 17 illustrate exemplary diagrams for explaining a method of calculating mapping loss according to an embodiment of the invention.

FIG. 18 illustrates an exemplary diagram for explaining a method of calculating integrated loss according to an embodiment of the invention.

FIG. 19 illustrates a block flow diagram for explaining a method for learning a 3D geometric structure of a molecule and a target property prediction method including the same, according to an embodiment of the invention.

FIG. 20 illustrates an example conceptual diagram for explaining first pre-training according to an embodiment of the invention.

FIG. 21 illustrates an example conceptual diagram for explaining second pre-training according to an embodiment of the invention.

FIG. 22 illustrates an example diagram for explaining a method for determining a model parameter freezing range for second pre-training according to an embodiment of the invention.

FIG. 23 illustrates an example conceptual diagram for explaining third pre-training according to an embodiment of the invention.

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

As is customary in the field, some embodiments are described and illustrated in the accompanying drawings in terms of functional blocks, units, and/or modules. Those skilled in the art will appreciate that these blocks, units, and/or modules are physically implemented by electronic (or optical) circuits, such as logic circuits, discrete components, microprocessors, hard-wired circuits, memory elements, wiring connections, and the like, which may be formed using semiconductor-based fabrication techniques or other manufacturing technologies. In the case of the blocks, units, and/or modules being implemented by microprocessors or other similar hardware, they may be programmed and controlled using software (e.g., microcode) to perform various functions discussed herein and may optionally be driven by firmware and/or software. It is also contemplated that each block, unit, and/or module may be implemented by dedicated hardware, or as a combination of dedicated hardware to perform some functions and a processor (e.g., one or more programmed microprocessors and associated circuitry) to perform other functions. Also, each block, unit, and/or module of some embodiments may be physically separated into two or more interacting and discrete blocks, units, and/or modules without departing from the scope of the inventive concepts. Further, the blocks, units, and/or modules of some embodiments may be physically combined into more complex blocks, units, and/or modules without departing from the scope of the inventive concepts.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

As the invention may make various changes and have several embodiments, specific embodiments will be illustrated in a drawing and described in a detailed description. Advantages and features of the invention and methods for achieving them will be made clear from the embodiments described below in detail with reference to the accompanying drawings. The invention may, however, be embodied in many different forms and should not be construed as being limited to the embodiments set forth herein. In the following embodiments, terms such as “first”, “second”, etc., are used to distinguish one component from another component rather than for a restrictive meaning. Singular expressions are intended to include plural expressions unless the context clearly indicates otherwise. Terms such as “include”, “comprise”, or “have” indicate the presence of features or components described in the specification, but do not preclude the possibility of addition of one or more other features or components. In the drawings, the sizes of components may be exaggerated or reduced for convenience of explanation. For example, the sizes and thicknesses of the components shown in the drawings are arbitrarily shown for convenience of explanation, and thus the invention is not necessarily limited to those shown in the drawing.

Hereinafter, embodiments of the invention will be described in detail with reference to the accompanying drawings. When described with reference to the drawings, identical or corresponding components will be given the same reference numerals, and redundant description of these components will be omitted.

[Exemplary System that Implements Model Encoder Training Service]

Hereinafter, an exemplary system that provides a model encoder training service in which a two-dimensional data-based encoder is pre-trained based on a denoise and distill (D&D) methodology using three-dimensional data will be described in detail with reference to the accompanying drawings.

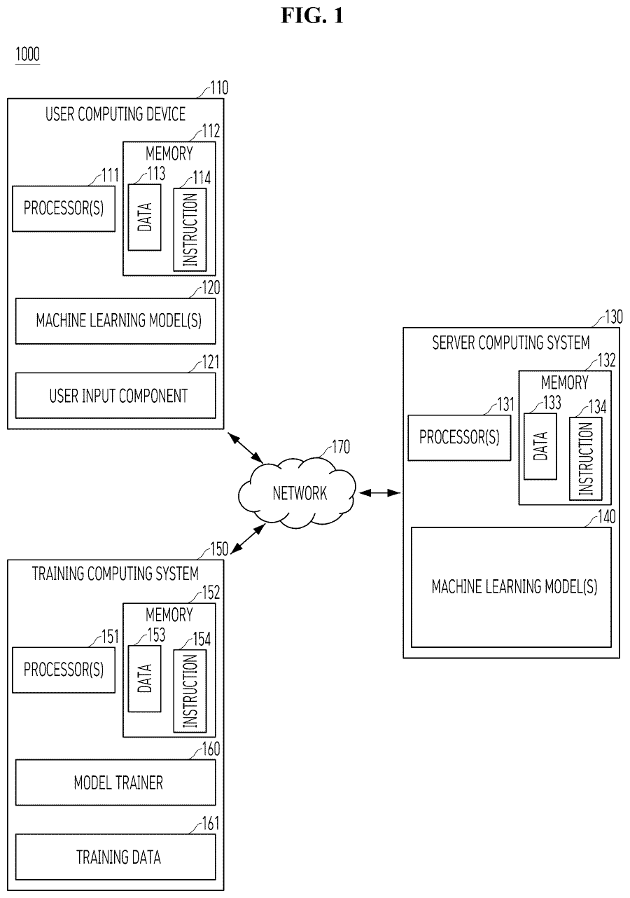

FIG. 1 illustrates an example block diagram of a computing system implementing a model encoder training service according to an embodiment of the invention.

Referring to FIG. 1, a computing system 1000 of the invention that implements a model encoder training service includes a user computing device 110, a server computing system 130, and a training computing system 150, and devices may communicate via a network 170.

A multitasking model training method according to an embodiment of the invention and a method for performing multitasking using a machine learning model trained based on this method 1) may be implemented and provided locally by the user computing device 110, 2) may be implemented and provided in the form of a web service by the server computing system 130 communicating with the user computing device 110, or 3) may be implemented and provided through interoperation between the user computing device 110 and the server computing system 130.

In this case, in the embodiment, the user computing device 110 and/or the server computing system 130 may train a machine learning model 120 and/or 140 through interaction with the training computing system 150 connected communicatively via the network 170. The training computing system 150 may be separate from the server computing system 130 or may be a part of the server computing system 130.

Also, in this instance, an artificial intelligence model may be 1) directly trained locally by the user computing device 110, 2) trained through interaction between the server computing system 130 and the user computing device 110 via the network 170, or 3) trained by a separate training computing system 150 using various training and learning techniques. In addition, the artificial intelligence model trained by the training computing system 150 may be implemented in such a manner as to be provided/updated by being transmitted via the network 170 to the user computing device 110 and/or the server computing system 130.

In some embodiments, the training computer system 150 may be a part of the server computing system 130 or a part of the user computing device 110.

The user computing device 110 may include all types of computing devices, such as smart phone, mobile phone, digital broadcasting device, personal digital assistant (PDA), portable multimedia player (PMP), desktop computer, wearable device, embedded computing device, and/or tablet PC.

Such a user computing device 110 includes at least one processor 111 and memory 112. The processor 111 may include at least one of a central processing unit (CPU), graphics processing unit (GPU), application-specific integrated circuits (ASICs), digital signal processors (DSPs), digital signal processing devices (DSPDs), programmable logic devices (PLDs), field-programmable gate arrays (FPGAs), controllers, micro-controllers, microprocessors, and/or other electrical units for performing functions or a plurality of electrically connected processors.

The memory 112 may include one or more non-transitory/transitory computer-readable storage media such as RAM, ROM, EEPROM, EPROM, flash memory devices, and magnetic disks, and combinations thereof, and may include web storage of a server that performs a storage function of the memory on the internet. Such memory 112 may store data 113 and instructions 114 required for the at least one processor 111 to perform a functional operation such as training an artificial intelligence model or executing multitasking learning through the artificial intelligence model.

In one embodiment, the user computing device 110 may store at least one machine learning model 120.

In detail, the machine learning model 120 may be various machine learning models such as a plurality of neural networks (e.g., deep neural networks), or other types of machine learning models including nonlinear models and/or linear models, and may be configured as a combination of them.

In this case, the neural network may include at least one of feed-forward neural networks, recurrent neural networks (e.g., long short-term memory recurrent neural networks), convolutional neural networks, and/or other forms of neural networks.

In one embodiment, the user computing device 110 may receive at least one machine learning model 120 from the server computing system 130 via the network 170, store it in the memory 112, and execute the stored machine learning model 120 by the processor 111 to perform multitask learning or the like.

In another embodiment, the server computing system 130 may include at least one machine learning model 140 to perform operations through the machine learning model 140, and may provide a model encoder training service to the user by interoperating with the user computing device 110 in such a manner that communicates related data to the user computing device 110.

For example, the user computing device 110 may perform the model encoder training service, in a manner where the server computing system 130 provides output in response to the user's input via the web by using the machine learning model 140.

Moreover, the artificial intelligence model may be implemented such that at least some of the machine learning models 120 and/or 140 are executed on the user computing device 110, while the rest are executed on the server computing system 130.

Furthermore, the user computing device 110 may include at least one input component 121 for detecting user input. For example, the user input component 121 may include a touch sensor (e.g., a touchscreen and/or touchpad) that detects touch from the user's input medium (e.g., a finger or stylus), an image sensor that detects the user's motion input, a microphone that detects the user's voice input, buttons, a mouse, and/or a keyboard. In addition, the user input component 121 may include an interface and an external controller (e.g., a mouse and/or keyboard) in cases where input from the external controller is received via the interface.

The server computing system 130 includes at least one processor 131 and memory 132. Here, the processor 131 may include at least one of a central processing unit (CPU), graphics processing unit (GPU), application-specific integrated circuits (ASICs), digital signal processors (DSPs), digital signal processing devices (DSPDs), programmable logic devices (PLDs), field-programmable gate arrays (FPGAs), controllers, micro-controllers, microprocessors, and/or other electrical units for performing functions, or a plurality of electrically connected processors.

The memory 132 may include one or more non-transitory and/or transitory computer-readable storage media, such as RAM, ROM, EEPROM, EPROM, flash memory devices, and magnetic disks, and combinations thereof. Such memory 132 may store data 133 and instructions 134 required for the processor 131 to perform a functional operation such as training an artificial intelligence model or executing multitask learning through the artificial intelligence model.

In one embodiment, the server computing system 130 may be implemented to include at least one computing device. For example, the server computing system 130 may be implemented to operate a plurality of computing devices according to a sequential computing architecture, a parallel computing architecture, or a combination thereof. Additionally, the server computing system 130 may include a plurality of computing devices connected via the network 170.

The server computing system 130 may store at least one machine learning model 140. For example, the server computing system 130 may include, as the machine learning model 140, a neural network and/or other multi-layer nonlinear models. Exemplary neural networks may include feed-forward neural networks, deep neural networks, recurrent neural networks, and convolutional neural networks.

The training computing system 150 includes at least one processor 151 and memory 152. The processor 151 may include at least one of a central processing unit (CPU), graphics processing unit (GPU), application-specific integrated circuits (ASICs), digital signal processors (DSPs), digital signal processing devices (DSPDs), programmable logic devices (PLDs), field-programmable gate arrays (FPGAs), controllers, micro-controllers, microprocessors, and/or other electrical units for performing functions, or a plurality of electrically connected processors.

The memory 152 may include one or more non-transitory and/or transitory computer-readable storage media, such as RAM, ROM, EEPROM, EPROM, flash memory devices, and magnetic disks, and combinations thereof. Such memory 152 may store data 153 and instructions 154 required for the processor 151 to perform a functional operation such as training an artificial intelligence model.

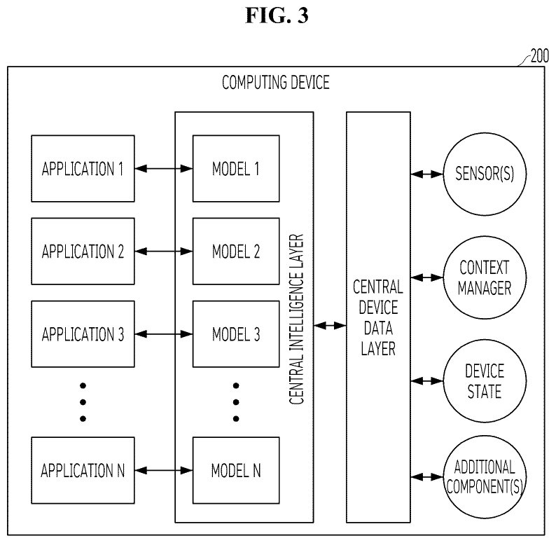

For example, the training computing system 150 may include a model trainer 160 that trains the machine learning model(s) (120 and/or 140) stored in the user computing device 110 and/or the server computing system 130, by using various training or learning techniques such as error backpropagation (in accordance with the framework illustrated in FIG. 3).

By way of example, such a model trainer 160 may perform updates to one or more parameters of the machine learning model(s) (120 and/or 140) through back-propagation based on a defined loss function.

In some implementation examples, performing error backpropagation may include performing truncated backpropagation through time. The model trainer 160 may perform a number of generalization techniques (e.g., weight decay, dropout, and/or knowledge distillation) to enhance the generalization capability of the machine learning model(s) (120 and/or 140) being trained.

Particularly, the model trainer 160 may train the machine learning model(s) 120 and/or 140 based on a set of training data 161. Here, the training data 161 may include different forms of data, such as images, audio samples, and/or text, for example. Examples of image types that can be used include video frames, LiDAR point clouds, X-ray images, computed tomography (CT) scans, hyperspectral images, and/or various other forms of images.

Such training data 161 may be provided by the user computing device 110 and/or the server computing system 130. When the training computing device trains the machine learning model(s) 120 and/or 140 on specific data from the user computing device 110, the machine learning model(s) 120 and/or 140 may be characterized as a personalized model.

Additionally, the model trainer 160 includes computer logic utilized to provide desired functionality.

Moreover, the model trainer 160 may be implemented in hardware, firmware, and/or software that controls a general-purpose processor. In one implementation example, the model trainer 160 may include program files stored on a storage device, loaded into the memory 152, and executed by one or more processors 151. In another implementation example, the model trainer 160 may include one or more sets of computer-executable data 153 and instructions 154 stored on a tangible computer-readable storage medium, such as RAM, hard disks, or optical or magnetic media.

The network 170 may include, but is not limited to, 3GPP (3rd Generation Partnership Project) network, LTE (Long Term Evolution) network, WiMAX (World Interoperability for Microwave Access) network, Internet, LAN (Local Area Network), Wireless LAN (Wireless Local Area Network), WAN (Wide Area Network), PAN (Personal Area Network), Bluetooth network, satellite broadcasting network, analog broadcasting network, and/or DMB (Digital Multimedia Broadcasting) network.

In general, communication via the network 170 may be performed using any type of wired and/or wireless connection, through various communication protocols (e.g., TCP/IP, HTTP, SMTP, and/or FTP), encodings or formats (e.g., HTML and/or XML), and/or protection schemas (e.g., VPN, secure HTTP, and/or SSL).

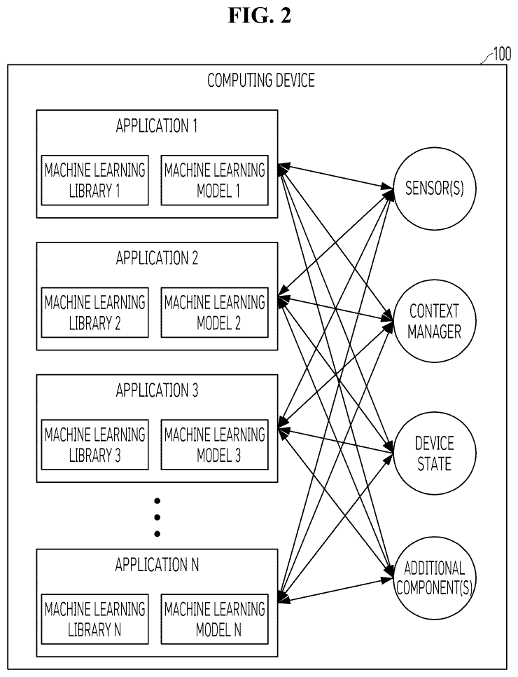

FIG. 2 illustrates an example block diagram of a computing device implementing a model encoder training service according to one embodiment of the invention.

Referring to FIG. 2, the computing device 100 included in the user computing device 110, the server computing system 130, and the training computing system 150 may include multiple applications (e.g., Application 1 through Application N). Each application may include a machine learning library and one or more machine learning models. For example, the applications may include image processing (e.g., detection, classification, and/or segmentation) applications, text messaging applications, email applications, dictation applications, virtual keyboard applications, browser applications, and/or chat-bot applications.

In the embodiment, the computing device 100 may include a model trainer 160 for training an artificial intelligence model, and may provide output data based on predetermined input data (e.g., material unique characteristic information and/or material property-specific information).

Each application of the computing device 100 may communicate with multiple other components of the computing device, such as at least one sensor, a context manager, a device state component, and/or additional components, for example. In one embodiment, each application may use an Application Programming Interface (API) (e.g., a public API) to communicate with each device component. In one embodiment, the API used by each application may be specific to that application.

FIG. 3 illustrates an example block diagram of another aspect of a computing device implementing a model encoder training service according to one embodiment of the invention.

Referring to FIG. 3, the computing device 200 includes multiple applications (e.g., Application 1 through Application N). Each application may communicate with a central intelligence layer. For example, the applications may include image processing applications, text messaging applications, email applications, dictation applications, virtual keyboard applications, and/or browser applications. In one embodiment, each application may use an API (e.g., a common API shared across all applications) to communicate with the central intelligence layer (and the models stored therein).

The central intelligence layer may include multiple machine learning models. For example, as illustrated in FIG. 3, at least some of the machine learning models may be provided for each application and managed by the central intelligence layer. In another implementation example, two or more applications may share a single machine learning model. For example, in some implementation examples, the central intelligence layer may provide a single model for all applications. In some implementation examples, the central intelligence layer may be incorporated into the operating system of the computing device 200 or implemented differently.

The central intelligence layer may communicate with a central device data layer. The central device data layer may serve as a centralized data storage location for the computing device 200. As illustrated in FIG. 3, the central device data layer may communicate with multiple other components of the computing device 200, such as one or more sensors, a context manager, a device state component, and/or additional components. In some implementation examples, the central device data layer may use an API (e.g., a private API) to communicate with each device component.

The technology described herein may make reference to servers, databases, software applications, and other computer-based systems, as well as actions taken and information transmitted to or from the above systems. It will be appreciated that the inherent flexibility of computer-based systems allows for a wide range of possible configurations, combinations, divisions of tasks, and functionality between and from components. For example, the processes described herein may be implemented using a single device or component, or multiple devices or components working in combination. Databases and applications may be implemented on a single system or distributed across multiple systems. Distributed components may operate sequentially or in parallel.

### [Multi-Tasking Learning Model (MtLM)]

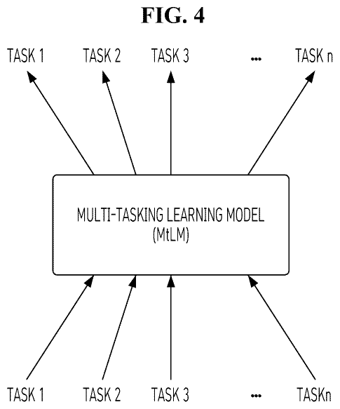

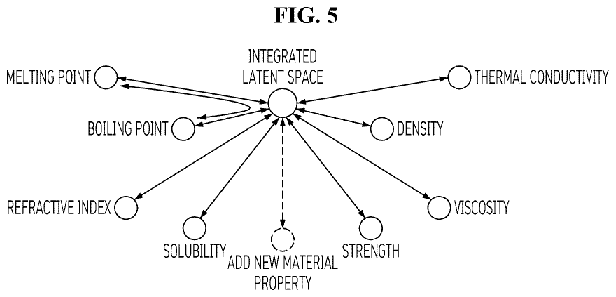

FIGS. 4 and 5 illustrate example conceptual diagrams for explaining a multitasking learning model (MtLM) according to one embodiment of the invention.

Referring to FIGS. 4 and 5, the multitasking learning model (MtLM) (Geometrically Aligned Transfer Encoder Model) according to the embodiment of the invention may be a machine learning model that aligns knowledge data (e.g., latent vectors) fragmented across task-specific latent spaces into one unified latent space (M: Manifold) through geometric transfer, in order to process multiple tasks for integrated output that satisfy a plurality of domains.

That is, the multitasking learning model (MtLM) according to the embodiment not only learns knowledge data across various domains simultaneously, but also efficiently learn relationships between multiple domains, thereby performing effective multitasking learning that expands the learning scope and at the same time implements batch learning of domain-specific local patterns and common principles between a plurality of domains.

Accordingly, the multitasking learning model (MtLM) may directly enhance the processing performance and accuracy of various multitasking tasks based on this model trained as above.

In the embodiment, the multitasking learning model (MtLM) will be described by using relationships between a material and a plurality of properties as an example, and a description will be given below of a learning model that performs multitasking of a plurality of tasks including a first task for predicting the characteristics of a first property of the material, a second task for predicting the characteristics of a second property of the material, and so on. However, it goes without saying that the invention is not limited to a learning method for multitasking relationships between a material and a plurality of properties and a prediction method, and may apply to various tasks that require a plurality of tasks to be simultaneously performed, such as relationships between a material and a plurality of properties.

In the embodiment, such a multitasking learning model (MtLM) may be pre-trained based on predetermined experimental data.

Here, the experimental data according to the embodiment is training data used to train the multitasking learning model (MtLM), and may be data including predetermined material unique characteristic information and material property-specific information.

In this case, the material unique characteristic information according to the embodiment may be information that specifies unique characteristics of a predetermined material.

For example, the material unique characteristic information may include at least one of a predetermined material's name, molecular structural formula, and/or chemical formula.

Moreover, the material property-specific information according to the embodiment may be information that specifies a predetermined material's data value for a predetermined property.

For example, the material property-specific information may include a predetermined material's property (i.e., domain) values such as boiling point, melting point, refractive index, solubility, viscosity, surface tension, density, strength, and/or thermal conductivity.

In the embodiment, the multitasking learning model (MtLM) pre-trained as described above may receive predetermined material unique characteristic information and/or material property-specific information as input and output predicted data based on the input information and learned knowledge.

In the embodiment, the multitasking learning model (MtLM) may receive predetermined material unique characteristic information as input and output predicted material property-specific information based on the input information and learned knowledge.

In another embodiment, the multitasking learning model (MtLM) may receive predetermined material property-specific information as input and output predicted material unique characteristic information based on the input information and learned knowledge.

In yet another embodiment, the multitasking learning model (MtLM) may include a model that is reverse-engineered to receive predetermined material unique characteristic information and material property-specific information as input and to output predicted optimal material unique characteristic information and material property-specific information based on the input information and learned knowledge.

In the following embodiment, molecular structural formula data representing material unique characteristic information will be used as input data, and characteristic value data of properties for each task will be used as output data.

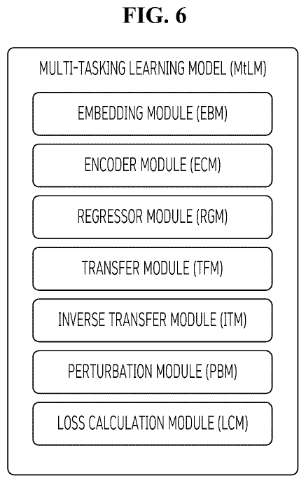

FIG. 6 illustrates an internal block diagram of a multitasking learning model (MtLM) according to an embodiment of the invention.

Referring to FIG. 6, in another aspect, the multitasking learning model (MtLM) according to the embodiment may include at least one embedding module (EBM), an encoder module (ECM), a regressor module (RGM), a transfer module (TFM), an inverse transfer module (ITM), a perturbation module (PBM), and a loss calculation module (LCM).

In detail, the embedding module (EBM) according to the embodiment of the invention may be a pre-encoder module that converts predetermined input data into an embedding vector.

Specifically, the embedding module may compress molecular structural formula data which is higher-dimensional data into an embedding vector which is a lower-dimensional representation, to reduce the dimensionality of the input to be processed by the encoder, thereby improving computational efficiency and learning speed and allowing the encoder to focus on and learn key features in a molecular structural formula for a pre-training task.

Through this, useful features in a model trained for a source task are easily applicable to a model to be trained for a target task, and overlapping features between different domains may be generalized, enabling effective distillation training for a new domain/task.

That is, the embedding module (EBM) may be a module that projects specific input data into a predetermined embedding space and converts it into a vector format.

In the embodiment, a graph neural network (GNN), which is suited for extracting molecular structural formula features, may be used as the embedding module (EBM), and for example, an embedding vector for input data may be provided based on a directed message passing neural network (DMPNN) architecture.

Additionally, the encoder module (ECM) according to the embodiment of the invention may be a module that receives a predetermined embedding vector as input and projects the input embedding vector into a latent space corresponding to the given task and converts it into a latent vector.

That is, the encoder module (ECM) may be a module that extracts key features from the input embedding vector and represents them in a corresponding latent space. In detail, the encoder module (ECM) may extract important features among the features of the embedding vector and perform data compression, such as removing unnecessary information or noise, by compressing data into a lower-dimensional space, thereby outputting a latent vector as a representation in the latent space.

In the embodiment, such an encoder module (ECM) may include a plurality of encoder modules respectively corresponding to a plurality of domains.

In the embodiment, the encoder module (ECM) may include a first encoder module (ECM) corresponding to a first domain (e.g., boiling point) and a second encoder module (ECM) corresponding to a second domain (e.g., melting point).

In another embodiment, the encoder module (ECM) may include a third encoder module (ECM) for performing a first task that predicts solubility in a first solvent and a fourth encoder module (ECM) for performing a second task that predicts solubility in a second solvent, which correspond to a third domain (e.g., solubility). That is, in another embodiment, multitasking may be performed for different tasks for the same domain. Naturally, a multitasking model that integrates multitasking for different domains and multitasking for different tasks within the same domain may also be included as one embodiment of the invention.

The following description will be given on the assumption that different domains are different tasks.

In this case, in the embodiment, one encoder module (ECM) among the plurality of encoder modules (ECM) may be a source encoder module (ECM) which is an encoder module (ECM) corresponding to the source task in distillation training according to the embodiment of the invention.

Moreover, one encoder module (ECM) among the remaining encoder modules (ECM), excluding the source encoder module (ECM), may be a target encoder module (ECM) which is an encoder module (ECM) corresponding to the target task in distillation training according to the embodiment of the invention.

In addition, the regressor module (RGM) according to the embodiment of the invention may be a head module that receives a predetermined latent vector as input and generates a final predicted value based on the input latent vector. That is, in the embodiment, the regressor module is used as an example of the head module.

This regressor module (RGM) is directly involved in generating the final output and may determine the prediction performance of the model.

Furthermore, in the embodiment, the regressor module (RGM) may include a plurality of regressor modules (RGMs) respectively corresponding to a plurality of domains.

In the embodiment, the regressor module (RGM) may include a first regressor module (RGM) corresponding to a first domain (e.g., boiling point) and a second regressor module (RGM) corresponding to a second domain (e.g., melting point).

In this case, in the embodiment, one regressor module (RGM) among the plurality of regressor modules (RGMs) may be a source regressor module (RGM) which is a regressor module (RGM) corresponding to the source task in distillation training according to the embodiment of the invention.

Moreover, one regressor module (RGM) among the remaining regressor modules (RGMs), excluding the source regressor module (RGM), may be a target regressor module (RGM) which is a regressor module (RGM) corresponding to the target task in distillation training according to the embodiment of the invention.

In addition, the transfer module (TFM) according to the embodiment of the invention may be a module that maps a predetermined latent vector to the latent space of another task and converts it into a transfer vector.

In detail, in the embodiment, the transfer module (TFM) may convert a specific latent vector into a transfer vector by mapping it to the latent space of another task based on Riemannian geometry.

In this process, the transfer module (TFM) may implement geometric alignment between the tasks to be mapped according to the embodiment of the invention. A detailed explanation of this will be provided below in the multitasking model training method.

That is, in the embodiment, the transfer module (TFM) can effectively perform knowledge data transfer across multiple tasks by mapping a latent vector of a first task to the latent space of a second task through geometric alignment according to the embodiment of the invention.

In this case, in the embodiment, the transfer module (TFM) may support data processing that enhances the accuracy and consistency of the converted vector (i.e., the transfer vector) by utilizing an autoencoder structure.

Further, in the embodiment, the transfer module (TFM) may include a plurality of transfer modules (TFMs) respectively corresponding to a plurality of domains.

In the embodiment, the transfer module (TFM) may include a first transfer module (TFM) corresponding to a first domain (e.g., boiling point) and a second transfer module (TFM) corresponding to a second domain (e.g., melting point).

In this case, in the embodiment, one transfer module (TFM) among the plurality of transfer modules (TFMs) may be a source transfer module (TFM) which is a transfer module (TFM) corresponding to the source task in distillation training according to the embodiment of the invention.

Moreover, one transfer module (TFM) among the remaining transfer modules (TFMs), excluding the source transfer module (TFM), may be a target transfer module (TFM) which is a transfer module (TFM) corresponding to the target task in distillation training according to the embodiment of the invention.

In addition, the inverse transfer module (ITM) according to the embodiment of the invention may be a module that reconstructs the transfer vector resulting from the mapping to the latent space of another task and conversion by the transfer module TFM, so that it is mapped back to the original latent space.

Accordingly, in the embodiment, the inverse transfer module (ITM) may generate a vector (hereinafter, inverse vector) by reconstructing the transfer vector and converting it back to the original one.

In this case, in the embodiment, the inverse transfer module (ITM) may enhance the stability of the above-described reconstruction process, as well as the accuracy and consistency of the transfer vector, by utilizing an autoencoder structure.

In the embodiment, such an inverse transfer module (ITM) may include a plurality of inverse modules (ITMs) respectively corresponding to a plurality of domains.

In the embodiment, the inverse transfer module (ITM) may include a first inverse module (ITM) corresponding to a first domain (e.g., boiling point) and a second inverse module (ITM) corresponding to a second domain (e.g., melting point).

In this case, in the embodiment, one inverse module (ITM) among the plurality of inverse modules (ITMs) may be a source inverse module (ITM) which is an inverse module (ITM) corresponding to the source task in distillation training according to the embodiment of the invention.

Moreover, one inverse module (ITM) among the remaining inverse modules (ITMs), excluding the source inverse module (ITM), may be a target inverse module (ITM) which is an inverse module (ITM) corresponding to the target task in distillation training according to the embodiment of the invention.

In addition, the perturbation module (PBM) according to the embodiment of the invention may be a module that generates a plurality of perturbation vectors by making some changes to a predetermined embedding vector.

In detail, in the embodiment, the perturbation module (PBM) may be a module that generates a plurality of perturbation vectors (i.e., perturbation points) in the vicinity of a particular embedding vector by making changes to the embedding vector to move it in a predetermined direction.

In this case, the plurality of perturbation vectors generated are designed to maintain a relative distance from the corresponding embedding vector, thereby effectively assisting in geometric alignment.

That is, the perturbation module (PBM) as described above may help align coordinate systems between the source task and the target task by generating a plurality of perturbation vectors to assist in the model's geometric alignment.

Moreover, in the embodiment, the perturbation module (PBM) may calculate the distance between a predetermined embedding vector and a plurality of perturbation vectors generated based on it, and may provide support to ensure that the source task and the target task have the same displacement based on the calculated distance.

Through this, the perturbation module (PBM) can more easily maintain the consistency of the model in the latent space.

In some embodiments, the perturbation module (PBM) may prevent model overfitting and improve generalization performance, by forcing a predetermined embedding vector and a plurality of perturbation vectors generated based on the embedding vector to maintain relationships.

In addition, the loss calculation module (LCM) according to the embodiment of the invention may be a module that calculates various loss functions based on various vectors obtained through the multitask learning model (MtLM).

In the embodiment, the loss calculation module (LCM) may calculate regression loss, autoencoder loss, consistency loss, mapping loss, distance loss, and/or integrated loss, according to the embodiment of the invention. A detailed explanation of this will be provided below in the multitasking model training method.

Through this, the loss calculation module (LCM) can support regularization and training of different parts of the model, and provide feedback for model training to achieve model optimization.

In the embodiment of the invention, the multitask learning model (MtLM) may perform model optimization and updates through various data processing processes working in conjunction with the above-described modules.

By way of example, the multitask learning model (MtLM) may perform model optimization and parameter updates by working in conjunction with the above-described modules based on an AdamW optimization algorithm.

As described above, in the embodiment of the invention, the multitask learning model (MtLM) not only learns knowledge data across various domains simultaneously, but also efficiently learns relationships between multiple domains, thereby performing effective multitasking learning that expands the learning scope and at the same time implements batch learning of domain-specific local patterns and common principles between a plurality of domains.

Accordingly, the multitasking learning model (MtLM) may directly enhance the processing performance and accuracy of various multitasking tasks based on this model trained as above.

[2D Graph Encoder (f_2D)]

A 2D graph encoder (f_2D) according to an embodiment of the invention may be an encoder that takes two-dimensional structural data of a molecule (hereinafter, 2D molecular data) as input.

This 2D graph encoder (f_2D) may serve as a student model that receives knowledge transferred (distilled) from a 3D conformer encoder described later. That is, the 2D graph encoder (f_2D) may act as an entity that receives knowledge transferred from the 3D conformer encoder.

Accordingly, the 2D graph encoder (f_2D) can accurately predict three-dimensional characteristic information using given 2D molecular data (e.g., a 2D molecular graph), even when three-dimensional structural data of the molecule (hereinafter, 3D molecular data) is insufficient.

Here, the three-dimensional characteristic information according to the embodiment may refer to information that specifies molecular characteristics provided through a molecular shape based on the 3D molecular data (e.g., molecular behavior pattern information, molecular physical force field information, etc.), among various properties (e.g., boiling point, melting point, surface tension, and/or solubility) of the molecule.

In detail, in the embodiment, the 2D graph encoder (f_2D) may learn a molecular structure based on 2D molecular data while following representations learned from the 3D conformer encoder.

Accordingly, even in the absence of 3D molecular data, the 2D graph encoder (f_2D) can predict the aforementioned three-dimensional characteristic information very accurately through the 2D molecular data.

In other words, in the embodiment, the 2D graph encoder (f_2D) can predict three-dimensional characteristic information with high accuracy based on given 2D molecular data through a process of learning representations similar to those of the 3D conformer encoder.

Through this, the 2D graph encoder (f_2D) can significantly reduce the data processing cost required to obtain three-dimensional characteristic information, while simultaneously improving the model's performance in various molecular/property prediction tasks.

More specifically, in the embodiment, the above 2D graph encoder (f_2D) may obtain 2D graph embeddings.

In detail, in the embodiment, the 2D graph encoder (f_2D) may receive 2D molecular data (e.g., a 2D molecular graph represented by predetermined nodes and edges) as input.

In addition, the 2D graph encoder (f_2D) may obtain embeddings for input nodes (e.g., atoms) and edges (e.g., bonds).

Moreover, in the embodiment, the 2D graph encoder (f_2D) may learn node-level and graph-level representations based on the obtained embeddings.

That is, the 2D graph encoder (f_2D) may learn the characteristics of each atom contained in the 2D molecular data, as well as the overall structural characteristics of the graph (e.g., a molecule or the like).

In addition, in the embodiment, the 2D graph encoder (f_2D) may learn interactions between each node in the 2D molecular data.

In the embodiment, the 2D graph encoder (f_2D) may learn interactions between each node using an attention mechanism.

Through this, the 2D graph encoder (f_2D) may infer complex interactions occurring in a 3D molecular structure through a 2D molecular graph.

From a structural perspective, the 2D graph encoder (f_2D) according to the embodiment may perform data processing and learning based on an attention-based architecture (for example, a TokenGT architecture or the like)

In the embodiment, the 2D graph encoder (f_2D) may include a TokenGT architecture, which is an attention-based architecture that provides maximum expressiveness for two-dimensional graphs.

Accordingly, the 2D graph encoder (f_2D) may generate consistent results regardless of the order of nodes and edges within the two-dimensional graph structure by utilizing all possible permutation-equivariant operators.

However, in the embodiment, the aforementioned attention-based architecture is not limited to the TokenGT architecture and may encompass various embodiments.

In the embodiment, the 2D graph encoder (f_2D) may implement an attention mechanism based on the above attention-based architecture.

In other words, by implementing the attention mechanism, the 2D graph encoder (f_2D) can learn interactions between nodes contained in the 2D molecular data, thereby enabling learning of structural features comparable to three-dimensional ones even in the absence of data on the 3D molecular structure.

In this case, in the embodiment, the 2D graph encoder (f_2D) may receive learned knowledge transferred from the 3D conformer encoder, based on D&D-GRAPH which is a graph-level D&D (Denoise and Distill) and D&D-NODE methodology which is a node-level D&D (Denoise and Distill).

Here, D&D-GRAPH according to the embodiment may be a method of transferring knowledge at the graph level through mean-pooled representations, which may be a learning method that reflects the overall structural characteristics of the graph.

Additionally, D&D-NODE according to the embodiment may be a method for performing fine-grained knowledge distillation at the node level by minimizing the differences between node representations, which may be a learning method that reflects the characteristics of each atom more precisely.

As described above, the 2D graph encoder (f_2D) according to the embodiment may receive learned knowledge transferred from the 3D conformer encoder based on a high-performance attention-based architecture (e.g., TokenGT or the like), thereby enabling highly accurate prediction of various molecular properties through 2D molecular data, even in the absence of three-dimensional structural information.

In some embodiments, the 2D graph encoder (f_2D) described above may be included in the above-described multitasking learning model or implemented and run on a separate external device and/or server.

In some embodiments, the above-described encoder module (ECM) of the multitasking learning model (MtLM) may be implemented based on the 2D graph encoder (f_2D) described above.

[3D Conformer Encoder (f_3D)]

According to an embodiment of the invention, the 3D conformer encoder (f_3D) may be an encoder that takes three-dimensional molecular data (i.e., three-dimensional structural data of a molecule) as input.

In the embodiment, the three-dimensional molecular data may include predetermined DFT data, etc.

For reference, the aforementioned DFT data may refer to computational data primarily obtained through density functional theory (DFT). Here, DFT is a quantum mechanical computational method which can be used to calculate the electronic structure of atoms and molecules. This DFT data utilizes the electron density of a material as a key variable to calculate the total energy of the system, thereby supporting the prediction of chemical/physical properties.

Returning to the discussion, the 3D conformer encoder (f_3D) described above may serve as a teacher model that performs pre-training using three-dimensional molecular data and transfers knowledge learned through the pre-training to the above-described 2D graph encoder (f_2D). That is, the 3D conformer encoder (f_3D) may be an entity that transfers knowledge to the 2D graph encoder (f_2D).

Accordingly, the 3D conformer encoder (f_3D) enables the 2D graph encoder (f_2D) to predict three-dimensional characteristic information with high accuracy through two-dimensional molecular data, even without explicit information about the 3D molecular structure.

In detail, in the embodiment, the 3D conformer encoder (f_3D) may receive three-dimensional molecular data as input.

Also, the 3D conformer encoder (f_3D) may obtain embeddings for nodes (e.g., atoms) and edges (e.g., bonds) in the input 3D molecular data.

In the embodiment, the 3D conformer encoder (f_3D) may perform pre-training using 3D molecular data based on the obtained embeddings.

In more detail, the 3D conformer encoder (f_3D) may perform pre-training to learn the spatial arrangement, etc. of nodes (e.g., atoms) and edges (e.g., bonds) in the three-dimensional molecular structure based on the 3D molecular data.

In this case, in the embodiment, the 3D conformer encoder (f_3D) may perform denoising-based learning.

Specifically, the 3D conformer encoder (f_3D) may receive, as input, 3D molecular data (hereinafter, 3D noising data) modified by adding artificial noise (e.g., Gaussian noise) to 3D molecular data.

Also, the 3D conformer encoder (f_3D) may perform learning on the three-dimensional molecular structure by removing noise from the input 3D noising data (that is, by restoring it to the original data).

In this case, in some embodiments, the 3D conformer encoder (f_3D) may perform learning while maintaining SE(3) invariance.

For reference, SE(3) is the abbreviation for the “Special Euclidean group in three dimensions,” which stands for a special Euclidean group in three-dimensional space. SE(3) refers to a set of transformations that includes rotations and translations in three-dimensional space, and may represent the position and orientation of objects in geometry and physics.

In the embodiment, the 3D conformer encoder (f_3D) may perform learning based on an SE(3) permutation invariant architecture (e.g., TorchMD-NET) which learns data representations invariant to rotations and translations.

That is, the 3D conformer encoder (f_3D) may perform learning using a neural network architecture with SE(3) invariance to extract and learn generalized knowledge that is robust to the relative position (arrangement) of a molecule in physical space.

Moreover, in the embodiment, the 3D conformer encoder (f_3D) may transfer (distill) the knowledge learned as above to the 2D graph encoder (f_2D).

In the embodiment, the 3D conformer encoder (f_3D) may transfer knowledge learned by pre-training to the 2D graph encoder (f_2D) based on cross-modal knowledge distillation.

As described above, the 3D conformer encoder (f_3D) according to the embodiment can effectively learn complex interactions in a three-dimensional molecular structure through a denoising approach and transfer learned knowledge to the 2D graph encoder (f_2D).

Through this, the 3D conformer encoder (f_3D) enables the 2D graph encoder (f_2D) to show strong performance in molecular property prediction tasks, even without explicit information about the three-dimensional molecular structure.

In some embodiments, the 3D conformer encoder (f_3D) described above may be included in the above-described multitasking learning model (MtLM), or may be implemented and run on a separate external device and/or server.

### [Method for Providing Multitasking Learning Model]

The following describes in detail a method in which a computing system 1000 according to an embodiment of the invention allows knowledge data in task-specific latent spaces to be mutually transferred and learned through geometric alignment in one unified latent space and, based on this, performs multitasking, in order to process multiple tasks for output across a plurality of domains.

The following describes in detail a method in which a computing system 1000 according to an embodiment of the invention allows knowledge data in task-specific latent spaces to be mutually transferred and learned through geometric alignment in one unified latent space and, based on this, performs multitasking, in order to process multiple tasks for output across a plurality of domains.

In general, the existing distillation training techniques have primarily focused on classification tasks involving image and/or language datasets, and they have limitations when solving regression problems or problems in non-Euclidean spaces.

In particular, when the training dataset is insufficient, the decline in prediction performance for the aforementioned problems becomes even more inevitable. Moreover, when multitasking across various task types is required, the learning and prediction performance for this is further degraded.

Additionally, most of the existing methods are optimized for handling data in Euclidean spaces, and therefore, they do not perform effectively in complex curved spaces or nonlinear spaces.

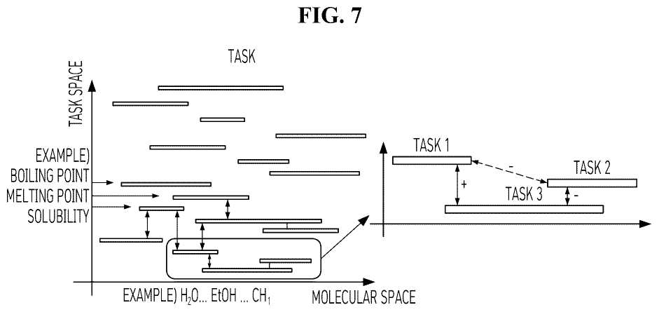

FIG. 7 illustrates an example conceptual diagram for explaining a multitasking model training method according to an embodiment of the invention.

Accordingly, as shown in FIG. 7, the computing system 1000 according to an embodiment of the invention aims to provide a novel multitasking model training method that can overcome regression problems with small-scale datasets and the limitations of the existing distillation training techniques, and a method for performing multitasking using a machine learning model trained based on the multitasking model training method.

In the following description of an embodiment of the invention, for effective explanation, the above-stated material is limited to a “molecules,” and the domain for this is explained based on “properties.”

This is based on the consideration that molecular datasets generally have small data volumes, encompass various task types, and primarily involve regression problems.

That is, in the case of molecular datasets, processing of various tasks associated with numerous properties is required, yet given data for this is very limited, and, each property tends to be closely interrelated or mutually influential.

Considering this, molecular datasets, which is advantageous for processing multiple tasks across a plurality of domains, may be a preferable example for describing a multitasking model training method and a method for performing multitasking using a machine learning model trained on this method, according to an embodiment of the invention.

However, this is not intended to be limiting, and it is obvious that any embodiment capable of applying multitasking across multiple domains may be included in the embodiment of the invention.

In the following, the multitasking model training method according to an embodiment of the invention and the method for performing multitasking using a machine learning model trained based on this method will be described in more detail with reference to the accompanying drawings.

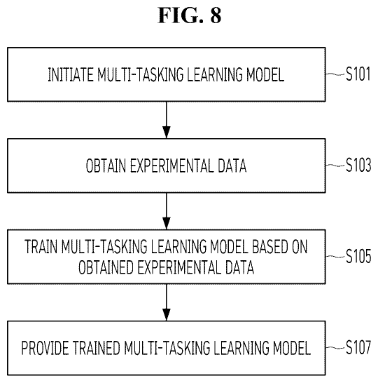

FIG. 8 illustrates a block flow diagram for explaining a multitasking model training method according to an embodiment of the invention.

Referring to FIG. 8, the multitask model training method according to an embodiment of the invention and the method for performing multitasking using a machine learning model trained based on this method may include the step (S101) of initializing a multitasking learning model (MtLM), the step (S103) of obtaining experimental data, the step (S105) of training the multitasking learning model (MtLM) based on the obtained experimental data, and the step (S107) of providing the trained multitasking learning model (MtLM).

In detail, the computing system 1000 according to an embodiment of the invention may initialize the multitasking learning model (MtLM). (S101)

Here, in other words, the multitasking learning model (MtLM) (Geometrically Aligned Transfer Encoder Model) may be a machine learning model that aligns knowledge data (e.g., latent vectors, etc.) fragmented across task-specific latent spaces into one unified latent space (M) through geometric transfer, in order to process multiple tasks for outputs across a plurality of domains.

That is, the multitask learning model (MtLM) according to the embodiment not only learns knowledge data across various domains simultaneously, but also efficiently learn relationships between multiple domains, thereby performing effective multitasking learning that expands the learning scope and at the same time implements batch learning of domain-specific local patterns and common principles between a plurality of domains.

In detail, in the embodiment, the computing system 1000 may perform initialization of each component included in the multitasking learning model (MtLM) described above.

In the embodiment, the computing system 1000 may initialize an embedding network (embedd(X)), encoder network (fₑ), regressor (head) network (fₕ), transfer network (fₜ), and/or inverse network (fᵢ) within the multitasking learning model (MtML) into random parameters (θ).

Moreover, in the embodiment, the computing system 1000 may also configure a predetermined optimization algorithm to be applied to the multitasking learning model (MtLM).

By way of example, the computing system 1000 may configure the AdamW (Decoupled Weight Decay Regularization) algorithm as an optimization algorithm, and, in some embodiments, may use the optimization algorithm by improving it so as to handle weight decay independently.

Additionally, the computing system 1000 according to an embodiment of the invention may obtain experimental data. (S103)

Here, in other words, the experimental data (X) according to the embodiment of the invention may be training data used for training the multitasking learning model (MtLM), and may include predetermined material unique characteristic information and material property-specific information.

In this case, the material unique characteristic information according to the embodiment may be information that specifies unique characteristics of a predetermined material. That is, in the embodiment, the material unique characteristic information may be information that specifies unique characteristics of a predetermined molecule.

For example, the material unique characteristic information may include a predetermined material's name, molecular structure, and/or chemical formula.

In addition, the material property-specific information according to the embodiment may refer to information that specifies data values of a predetermined material with respect to a predetermined property.

By way of example, the material property-specific information may include property (i.e., domain) values such as the boiling point, melting point, refractive index, solubility, viscosity, surface tension, density, strength, and/or thermal conductivity of a predetermined material.

In detail, in the embodiment, the computing system 1000 may obtain experimental data as described above, based on predetermined user input and/or interaction with an external server.

Furthermore, the computing system 1000 according to an embodiment of the invention may train the multitasking learning model (MtLM) based on the obtained experimental data. (S105)

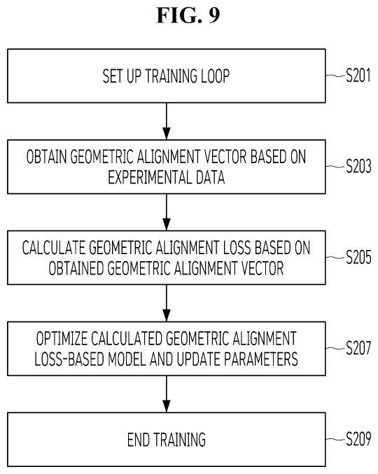

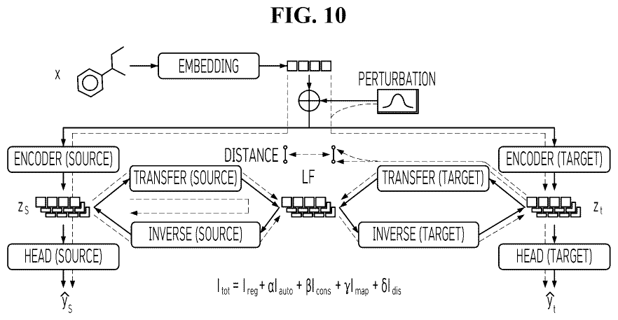

FIG. 9 illustrates a block flow diagram for explaining a method of training a multitasking learning model (MtLM) according to an embodiment of the invention, and FIG. 10 illustrates an example conceptual diagram for explaining a method of training a multitasking learning model (MtLM) according to an embodiment of the invention.

That is, referring to FIGS. 9 and 10, in the embodiment, the computing system 1000 may perform pre-training of the multitasking learning model (MtLM) based on the experimental data obtained as described above.

In detail, in the embodiment, the computing system 1000 may configure a training loop for the multitasking learning model (MtLM). (S201)

In more detail, in the embodiment, the computing system 1000 may set the number of repeats for an epoch, the number of repeats for a task, and/or the number of repeats for a batch, during training.

In the embodiment, the computing system 1000 may configure the training loop to repeat epoch ‘i’ from 1 to n (where n>=1), repeat each task ‘t’, and repeat each predetermined batch ‘b’ during training.

Moreover, in the embodiment, the computing system 1000 may obtain geometric alignment vectors based on the experimental data obtained as described above. (S203)

Here, the geometric alignment vectors according to the embodiment of the invention may refer to various vectors obtained through the multitasking learning model (MtLM).

In the embodiment, the geometric alignment vectors may include an embedding vector (a), a perturbation vector ({ā}), an encoding vector, a transfer vector, and an inverse vector.

In detail, in the embodiment, the computing system 1000 may input the obtained experimental data into the multitasking learning model (MtLM).

In addition, in the embodiment, the computing system 1000 may obtain 1) an embedding vector based on the multitasking learning model (MtLM) into which the experimental data has been inputted.

In more detail, the computing system 1000 may interoperate with the embedding module (EBM) of the multitasking learning model (MtLM) to convert the input experimental data into an embedding vector via an embedding network.

Accordingly, the computing system 1000 may obtain an embedding vector by projecting the experimental data into a predetermined embedding space and converting it into a vector format.

In addition, in the embodiment, the computing system 1000 may generate 2) a perturbation vector based on the obtained embedding vector.

In detail, in the embodiment, the computing system 1000 may interoperate with the perturbation module (PBM) of the multitasking learning model (MtLM) to generate a plurality of perturbation vectors (i.e., perturbation points) in a predetermined vicinity of the obtained embedding vector.

In this case, in the embodiment, the computing system 1000 may repeatedly perform the above-described functional operation for each task to obtain a perturbation vector corresponding to each task.

In the embodiment, the computing system 1000 may obtain a perturbation vector corresponding to task ‘t’ and a perturbation vector corresponding to task ‘s’.

Furthermore, in the embodiment, the computing system 1000 may obtain 3) an encoding vector based on the generated perturbation vector and embedding vector.

Here, the encoding vector according to the embodiment may include a perturbation latent vector, which is a latent vector generated based on a predetermined perturbation vector, and an original latent vector, which is generated based on the embedding vector which is the original vector of the perturbation vector.

In detail, in the embodiment, the computing system 1000 may interoperate with the encoder module of the multitasking learning model (MtLM) to project the generated perturbation vector into a latent space corresponding to the relevant task via an encoder network and convert it into a latent vector.

Furthermore, in the embodiment, the computing system 1000 may interoperate with the encoder module of the multitasking learning model (MtLM) to project the obtained embedding vector into a latent space corresponding to the relevant task via an encoder network and convert it into a latent vector.

Accordingly, in the embodiment, the computing system 1000 may obtain a perturbation latent vector and an original latent vector.

In this case, in the embodiment, the computing system 1000 may repeatedly perform the above-described functional operation for each task to obtain an original latent vector and perturbation latent vector corresponding to each task.

In the embodiment, the computing system 1000 may obtain an original latent vector (zₜ: hereinafter, t-th original latent vector) corresponding to task ‘t’ and a perturbation latent vector ({z̅ₜ}: hereinafter, t-th perturbation latent vector) corresponding to task ‘t’.

Also, the computing system 1000 may obtain an original latent vector (zₛ: hereinafter, s-th original latent vector) corresponding to task ‘s’ and a perturbation latent vector ({z̅ₛ}: hereinafter, s-th perturbation latent vector) corresponding to task ‘s’.

Furthermore, in the embodiment, the computing system 1000 may obtain 4) a transfer vector based on the obtained encoding vector.

Here, the transfer vector according to the embodiment may include a perturbation transfer vector, which is a transfer vector generated based on a predetermined perturbation latent vector, and an original transfer vector, which is a transfer vector generated based on the original latent vector corresponding to the perturbation latent vector.

In detail, in the embodiment, the computing system 1000 may interoperate with the transfer module (TFM) of the multitasking learning model (MtLM) to map the obtained perturbation latent vector and original latent vector into the latent space of another task (e.g., task ‘s’ or task ‘t’) via a transfer network and convert them into transfer vectors.

Accordingly, the computing system 1000 may obtain a perturbation transfer vector and an original transfer vector.

In this case, in the embodiment, the computing system 1000 may repeatedly perform the above-described functional operation for each task to obtain an original transfer vector and perturbation transfer vector corresponding to each task.

In the embodiment, the computing system 1000 may obtain an original transfer vector (mₜ: hereinafter, t-th original transfer vector) corresponding to task ‘t’ and a perturbation transfer vector ({m̅ₜ}: hereinafter, t-th perturbation transfer vector) corresponding to task ‘t’.

Also, the computing system 1000 may obtain an original transfer vector (mₛ: hereinafter, s-th original transfer vector) corresponding to task ‘s’ and a perturbation transfer vector ({m̅ₛ}: hereinafter, s-th perturbation transfer vector) corresponding to task ‘s’.

Accordingly, in the embodiment, the computing system 1000 may obtain geometric alignment vectors based on experimental data (i.e., an embedding vector, a perturbation vector, an encoding vector (including an original latent vector and a perturbation latent vector), and a transfer vector (including an original transfer vector and a perturbation transfer vector)).

In addition, in the embodiment, the computing system 1000 may obtain 5) an inverse vector based on the obtained transfer vector.

Here, the inverse vector according to the embodiment may include a perturbation inverse vector, which is an inverse vector generated based on a predetermined perturbation transfer vector, and an original inverse vector, which is an inverse vector generated based on the original transfer vector corresponding to the perturbation transfer vector.

In detail, in the embodiment, the computing system 1000 may interoperate with the inverse module (ITM) of the multitasking learning model (MtLM) to reconstruct the obtained perturbation transfer vector and original transfer vector via an inverse network and convert them into inverse vectors so as to be mapped back into the original latent space.

Accordingly, the computing system 1000 may obtain a perturbation inverse vector and an original inverse vector.

In this case, in the embodiment, the computing system 1000 may repeatedly perform the above-described functional operation for each task to obtain an original inverse vector and perturbation inverse vector corresponding to each task.

In the embodiment, the computing system 1000 may obtain an original inverse vector (\hat{z}ₜ: hereinafter, t-th original inverse vector) corresponding to task ‘t’ and a perturbation inverse vector

$$( {𝓏}_{t}^{′} :$$

hereinafter, t-th perturbation inverse vector) corresponding to task ‘t’.

Also, the computing system 1000 may obtain an original inverse vector (\hat{z}ₛ: hereinafter, s-th original inverse vector) corresponding to task ‘s’ and a perturbation inverse vector (zₛ′: hereinafter, s-th perturbation inverse vector) corresponding to task ‘s’.

Accordingly, in the embodiment, the computing system 1000 may obtain geometric alignment vectors based on experimental data (i.e., an embedding vector, a perturbation vector, an encoding vector (including an original latent vector and a perturbation latent vector), a transfer vector (including an original transfer vector and a perturbation transfer vector), and an inverse vector (including an original inverse vector and a perturbation inverse vector).

Moreover, in the embodiment, the computing system 1000 may calculate geometric alignment loss based on the obtained geometric alignment vectors. (S205)

Here, the geometric alignment loss according to the embodiment of the invention may refer to various loss functions calculated based on various vectors (i.e., geometric alignment vectors) obtained through the multitasking learning model (MtLM).

In the embodiment, the geometric alignment loss may include regression loss (L_{reg}), autoencoder loss (Lₐᵤₜₒ), consistency loss (L_{cons}), mapping loss (Lₘₐₚ), a distance loss (L_{dis}), and/or integrated loss (Lₜₒₜ).

In the following description, for the purpose of effective explanation, the geometric alignment loss is described as being calculated based on task ‘t’.

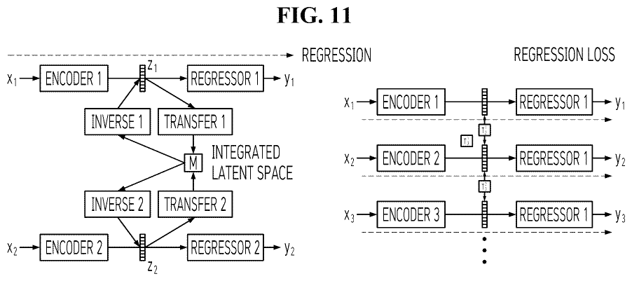

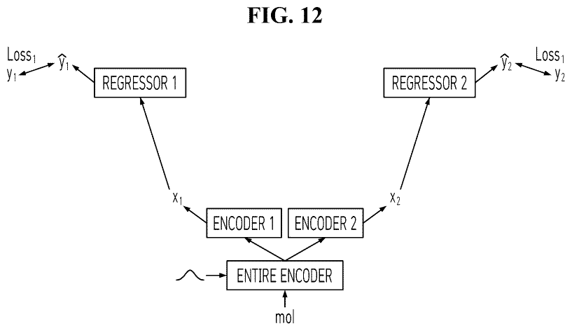

FIGS. 11 and 12 illustrate exemplary diagrams for explaining a method of calculating regression loss according to an embodiment of the invention.

In detail, referring to FIGS. 10 through 12, in the embodiment, the computing system 1000 may calculate 1) regression loss based on the multitasking learning model (MtLM) from which geometric alignment vectors have been obtained.

In more detail, in the embodiment, the computing system 1000 may calculate regression loss based on a predicted value ( [symbol] ₜ) predicted through the regressor module (RGM) and an actual value ( [symbol] _{y}, i.e., label value) according to the following [Mathematical Formula 1]. Here, the predicted value in [Mathematical Formula 1] may be represented as ‘fₕ(zₜ)’.

$${L}_{reg} = M S E ( {\hat{y}}_{t} , {y}_{t} ) \qquad [ Mathematical Formula 1 ]$$

That is, the computing system 1000 may calculate regression loss by computing the mean squared error (MSE) between the predicted value and the actual value.

In this case, in the embodiment, it is possible to prevent mutual interference between each task by calculating independent regression loss based on the encoder module (ECM) and regressor module (RGM) that matches each task and performing training based on this.

As such, the computing system 1000 can easily evaluate the model's regression performance by calculating regression loss.

Moreover, referring further to FIG. 10, in the embodiment, the computing system 1000 may calculate 2) autoencoder loss based on the multitasking learning model (MtLM) from which geometric alignment vectors have been obtained.

In detail, in the embodiment, the computing system 1000 may calculate autoencoder loss based on the original latent vector and the original inverse vector according to the following [Mathematical Formula 2].

$${L}_{auto} = M S E ( {\hat{𝓏}}_{t} , {𝓏}_{t} ) \qquad [ Mathematical Formula 2 ]$$

The computing system 1000 may calculate autoencoder loss by computing the mean squared error (MSE) between the latent vector and the inverse vector.

In the embodiment, the computing system 1000 may enhance the accuracy of the data transfer process through the autoencoder loss calculated as above.

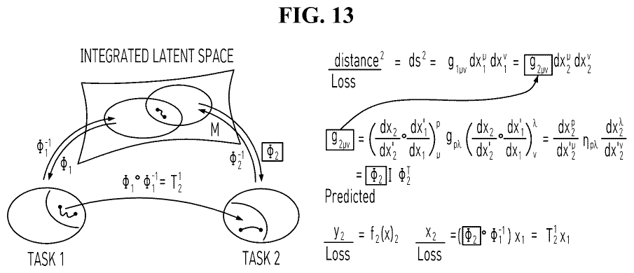

FIG. 13 illustrates an exemplary diagram for explaining a method of mapping to a unified latent space (M) according to an embodiment of the invention.

Referring to FIG. 13, in the embodiment, the computing system 1000 may learn a bidirectional transformation matrix (TM) that enables each task to be mapped to a common unified latent space (M).

In detail, in the embodiment, the computing system 1000 may connect latent spaces of tasks by utilizing knowledge data containing labels for both tasks.

In this process, the computing system 1000 may calculate consistency loss and mapping loss according to the embodiment.

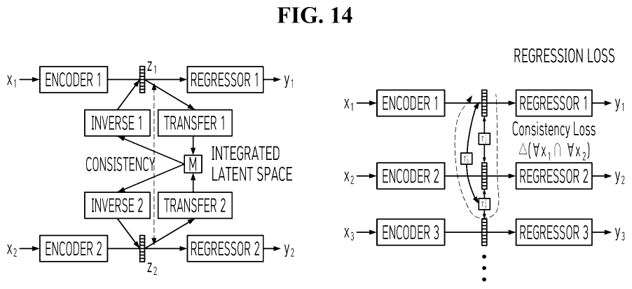

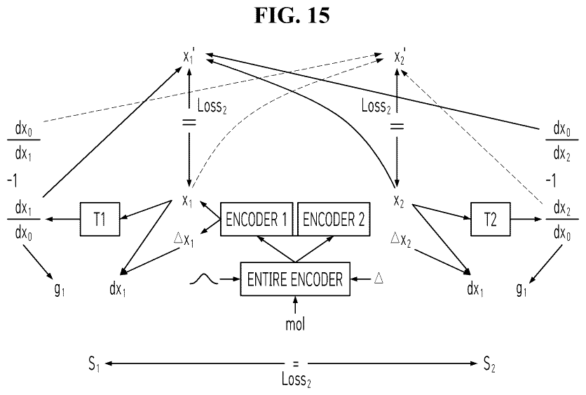

FIGS. 14 and 15 illustrate exemplary diagrams for explaining a method of calculating consistency loss according to an embodiment of the invention.

In more detail, referring to FIGS. 10, 14, and 15, in the embodiment, the computing system 1000 may calculate 3) consistency loss based on the multitasking learning model (MtLM) from which geometric alignment vectors have been obtained.

Specifically, in the embodiment, the computing system 1000 may calculate consistency loss based on the perturbation transfer vector of task ‘t’ and the perturbation transfer vector of task ‘s’ according to the following [Mathematical Formula 3].

$${L}_{cons} = M S E ( \{ {\overline{m}}_{s} \} , \{ {\overline{m}}_{t} \} ) \qquad [ Mathematical Formula 3 ]$$

That is, the computing system 1000 may calculate consistency loss by computing the mean squared error (MSE) between the t-th perturbation transfer vector and the s-th perturbation transfer vector.

In this case, in the embodiment, the computing system 1000 may derive a metric for calculating spatial distances from the transformation matrix (TM), and may perform training so as to make the distances equal within the latent spaces of each task based on the derived metric.

Through this, the computing system 1000 may more effectively implement geometric alignment between tasks.

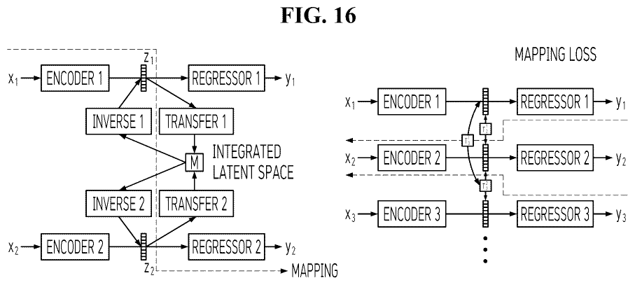

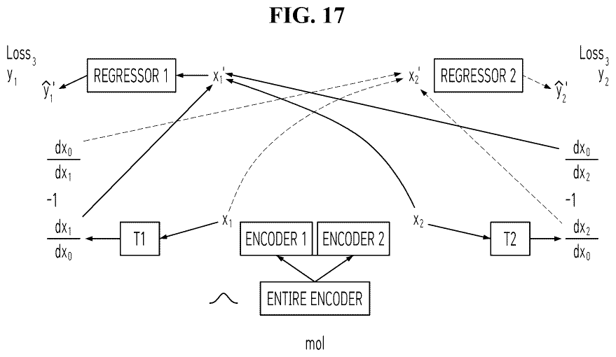

FIGS. 16 and 17 illustrate exemplary diagrams for explaining a method of calculating mapping loss according to an embodiment of the invention.

Referring to FIGS. 10, 16, and 17, in the embodiment, the computing system 1000 may calculate 4) mapping loss based on the multitasking learning model (MtLM) from which geometric alignment vectors have been obtained.

In detail, in the embodiment, the computing system 1000 may calculate mapping loss based on the actual value for task ‘t’ and the predicted value based on the original inverse vector of task ‘s’ according to [Mathematical Formula 4].

$${L}_{map} = M S E ( {f}_{h} ( {f}_{i} ( {m}_{s} ) ) , {y}_{t} ) \qquad [ Mathematical Formula 4 ]$$

That is, the computing system 1000 may calculate mapping loss by computing the mean squared error (MSE) between the actual value for task ‘t’ and the predicted value based on the original inverse vector of task ‘s’.

In the embodiment, by calculating mapping loss as described above, the computing system 1000 may implement training in such a manner as to transfer a latent vector from the latent space of one task to the latent space of another task and to perform the other task based on the transferred vector, thereby inducing mutual similarity between latent features.

Through this, the computing system 1000 may evaluate the prediction performance of a vector transferred to the latent space of another task and induce learning in a direction that improves such performance.

In addition, referring further to FIG. 10, in the embodiment, the computing system 1000 may calculate 5) distance loss based on the multitasking learning model (MtLM) from which geometric alignment vectors have been obtained.

In detail, in the embodiment, the computing system 1000 may calculate distance loss between tasks based on the distance (Sᵢ: hereinafter, transfer vector displacement) between the original transfer vector and perturbation transfer vector of each task, according to the following [Mathematical Formulae 5] and [Mathematical Formula 6].

In more detail, in the embodiment, the computing system 1000 may calculate the distance

$$( {s}_{i}^{t} :$$

hereinafter, t-th transfer vector displacement) between the t-th original transfer vector and t-th perturbation transfer vector of task ‘t’ according to the following [Mathematical Formula 5(a)].

Also, in the embodiment, the computing system 1000 may calculate the distance (

$${s}_{i}^{s} :$$

hereinafter, s-th transfer vector displacement) between the s-th original transfer vector and s-th perturbation transfer vector of task ‘s’ according to the following [Mathematical Formula 5(b)].

$$[ Mathematical Formulae 5 ]$$

$${s}_{i}^{s} = {m}_{t} - \{ {\overline{m}}_{t} \} \qquad ( a )$$

$${s}_{i}^{t} = {m}_{s} - \{ {\overline{m}}_{t} \} \qquad ( b )$$

Moreover, in the embodiment, the computing system 1000 may calculate distance loss by computing the mean squared error (MSE) between the t-th transfer vector displacement and the s-th transfer vector displacement according to [Mathematical Formula 6].

$${L}_{dis} = \frac{1}{M} {\sum }_{i} M S E ( {s}_{i}^{s} , {s}_{i}^{t} ) \qquad [ Mathematical Formula 6 ]$$

Here, ‘M’ in [Mathematical Formula 6] denotes the number of perturbation points.

In this case, in the embodiment, the computing system 1000 may define the t-th transfer vector displacement and the s-th transfer vector placement as the displacements for the source task and target task, respectively.

Accordingly, the computing system 1000 may interpret the t-th transfer vector displacement and the s-th transfer vector displacement as being on a flat Euclidean space, thereby more easily calculating the distance between the original transfer vector and the perturbation transfer vector.

Therefore, the computing system 1000 may keep the consistency of the model across latent spaces more intact.

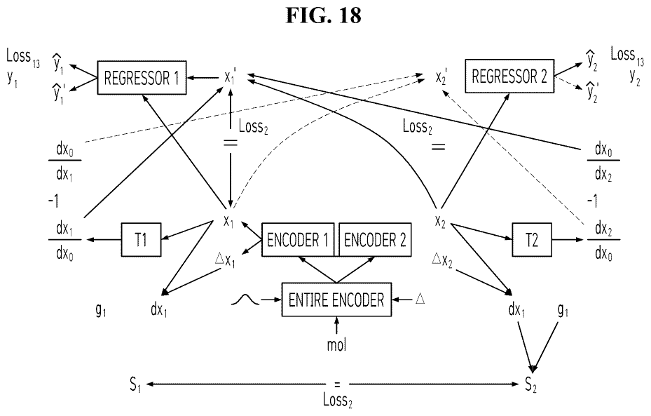

FIG. 18 illustrates an exemplary diagram for explaining a method of calculating integrated loss according to an embodiment of the invention.

Moreover, referring to FIGS. 10 and 18, in the embodiment, the computing system 1000 may calculate 6) integrated loss based on the multitasking learning model (MtLM) from which geometric alignment vectors have been obtained.

In detail, in the embodiment, the computing system 1000 may calculate integrated loss by weighted summation of the above-described regression loss, autoencoder loss, consistency loss, mapping loss, and distance loss according to the following [Mathematical Formula 7].

$${L}_{tot} = {L}_{reg} + α {L}_{auto} + β {L}_{cons} + γ {L}_{map} + δ {L}_{dis} \qquad [ Mathematical Formula 7 ]$$

In the embodiment, the computing system 1000 may apply a weight to each loss function so that each loss function can be optimized for a specific aspect of the model.

Here, in [Mathematical Formula 7], ‘α’ denotes the weight for the autoencoder loss, ‘β’ denotes the weight for the consistency loss, ‘γ’ denotes the weight for the mapping loss, and ‘δ’ denotes the weight for the distance loss.

In the embodiment, the computing system 1000 may utilize the aforementioned weights to adjust the importance of a loss function corresponding to each weight during the model training process, thereby updating parameters in a direction that minimizes the integrated loss.

Returning to FIG. 9, in the embodiment, the computing system 1000 may also perform model optimization and parameter updates based on the geometric alignment loss calculated as described above. (S207)

In detail, in the embodiment, the computing system 1000 may perform optimization and parameter updates for the multitasking learning model (MtLM) based on the above-described integrated loss.

In the embodiment, the computing system 1000 may calculate the gradient for each parameter of the multitasking learning model (MtLM) based on the integrated loss through backpropagation.

Also, the computing system 1000 may perform parameter updates for the multitasking learning model (MtLM) by using the calculated gradient, a preset optimization algorithm (e.g., the AdamW (Decoupled Weight Decay Regularization) algorithm, and so on.

Accordingly, the computing system 1000 can implement optimization of the multitasking learning model (MtLM) based on the geometric alignment loss (particularly, the integrated loss).

As such, in the embodiment, the computing system 1000 may perform optimization of the multitasking learning model (MtLM) and parameter update training through a combination of multiple loss functions calculated from various perspectives.

In this case, each loss function may easily assist in improving the model's performance by correcting for the accuracy, consistency, and/or distance in knowledge data mapping.

Through this, the computing system 1000 can implement a multitasking model that not only provides improved performance but also operates more stably and offers enhanced generalization performance, by overcoming regression problems in small-scale datasets and limitations of conventional distillation training techniques.

Additionally, in the embodiment, the computing system 1000 may terminate the training of the multitasking learning model (MtLM). (S209)

In detail, in an embodiment, the computing system 1000 may terminate the training process of the multitasking learning model (MtLM) once preset training termination conditions are satisfied.

In the embodiment, the computing system 1000 may terminate the training of the multitasking learning model (MtLM) upon completion of the configured training loop.

Returning to FIG. 8, the computing system 1000 according to an embodiment of the invention may also provide the trained multitasking learning model (MtLM). (S107)

That is, in the embodiment, the computing system 1000 may provide the multitasking learning model (MtLM) trained as described above in a predetermined manner.

In the embodiment, the computing system 1000 may provide the multitasking learning model (MtLM) trained according to the embodiment of the invention by interoperating with a predetermined application service (e.g., material synthesis/evaluation service, material property prediction service, and/or optimal material recommendation service).

Accordingly, the computing system 1000 can effectively support the processing of various multitasking tasks using a multitasking learning model (MtLM) with enhanced performance.

In this way, in the embodiment, the computing system 1000 may provide a multitasking learning model (MtLM) that provides improved performance and operates more stably that overcomes regression problems in small-scale datasets and limitations of conventional distillation training techniques, by allowing knowledge data in task-specific latent spaces to be mutually transferred and learned through geometric alignment in one unified latent space, in order to process multiple tasks for output across a plurality of domains.

Through this, the computing system 1000 can provide a distillation training-based multitasking model that shows high generalization performance and operates robustly and stably, even in situations where the model is given a small amount of data, encompasses various task types, or deals mainly with regression problems.

In other words, the computing system 1000 can provide a multitasking learning model (MtLM) with enhanced prediction performance based on knowledge distilled through geometric alignment-based distillation training which is performed in conjunction with other domains, even when there are domains lacking sufficient experimental data (training data), out of a plurality of domains (e.g., properties).

For example, the computing system 1000 may pre-train the multitasking learning model (MtLM) based on first to tenth properties for each of a plurality of molecular structural formulae, and thereafter, upon receiving a first molecular structural formula that only contains data for the first to fifth properties, may more accurately predict data values for the remaining sixth to tenth properties for the first molecular structural formula based on knowledge data transferred and distilled through pre-training and generate and provide output data based on the predictions.

Accordingly, the computing system 1000 according to the embodiment of the invention can implement effective distillation training based on geometric alignment, ensure high generalization performance, improve prediction accuracy for regression problems, support regularization through a combination of various loss functions, and perform a stable training process to provide a multitasking model that ensures robust performance.

As above, a multitasking model training method according to an embodiment of the invention and a method for performing multitasking using a machine learning model trained based on this method can provide a multitasking model that maintains high performance even with small-scale datasets, by overcoming the data scarcity problem by transferring knowledge learned from a source task to a target task through distillation training.

Therefore, the multitasking model training method according to an embodiment of the invention and the method for performing multitasking using a machine learning model trained based on this method can expand the range of application of machine learning models to areas where machine learning models were hard to apply due to a lack of data or domain knowledge.

Furthermore, the multitasking model training method according to an embodiment of the invention and the method for performing multitasking using a machine learning model trained based on this method can deliver high prediction performance even in complex regression problems such as molecular datasets, by providing a specialized distillation training technique that can be effectively applied to regression problems.

Furthermore, the multitasking model training method according to an embodiment of the invention and the method for performing multitasking using a machine learning model trained based on this method can maintain geometric consistency between tasks and enhance the efficiency of distillation training, by optimizing knowledge transfer between the source task and the target task through a Riemannian geometric approach.

Furthermore, the multitasking model training method according to an embodiment of the invention and the method for performing multitasking using a machine learning model trained based on this method can further enhance the model's generalization performance by combining multiple loss functions to regularize various aspects of the model.

Therefore, the multitasking model training method according to an embodiment of the invention and the method for performing multitasking using a machine learning model trained based on this method provide a multitasking model that can be universally applied to a variety of materials (substances), thereby contributing to improved quality across related industries.

### [Method for Learning 3D Geometric Structure of Molecule and Target Property Prediction Method Including the Same]

Hereinafter, a method for providing a model encoder training service, in which the computing system 1000 according to an embodiment of the invention performs pre-training of a 2D data-based encoder based on a D&D (Denoise and Distill) methodology using three-dimensional data, will be described in detail with reference to the accompanying drawings.

In general, the existing 2D molecular data-based learning techniques have limitations in that they are hard to achieve significant improvement in prediction performance due to distortions in molecular graphs that occur during data augmentation.

Moreover, the above-mentioned three-dimensional characteristic information (for example, molecular behavior pattern information, molecular physical force field information, etc.), among various properties of the molecule (e.g., boiling point, melting point, surface tension, and/or solubility), can serve as very important factors in processing various molecular/property prediction tasks. However, it is difficult to easily estimate and predict this information using the existing 2D molecular data-based learning methods.

In addition, the existing 3D molecular data-based learning techniques incur high computational costs since they require accurate coordinates of 3D molecular structures.

Therefore, the computing system 1000 according to an embodiment of the invention aims to provide a novel learning method (i.e., a D&D (Denoise and Distill) framework-based learning method that includes a 2D graph encoder (f_2D) and a 3D conformer encoder (f_3D)) capable of overcoming the performance limitations and data processing cost issues of the existing learning techniques.

Specifically, the computing system 1000 according to an embodiment of the invention aims to provide a 2D graph encoder (f_2D) that implements high-performance molecular property prediction without requiring explicit information on 3D molecular structures, by performing denoising-based training using 3D molecular data through a 3D conformer encoder (f_3D) to learn generalized knowledge based on 3D molecular structures, transferring the learned knowledge to the 2D graph encoder (f_2D), and optimizing the knowledge-distilled 2D graph encoder (f_2D) for target properties (e.g., boiling point, melting point, and/or solubility).

Likewise, in the following description of an embodiment of the invention, for effective explanation, the above material is limited to “molecules,” and the domain for this is explained based on “properties” but is not limited to this.

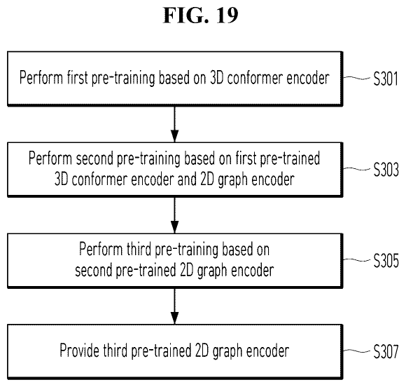

FIG. 19 illustrates a block flow diagram for explaining a method for learning a 3D geometric structure of a molecule and a target property prediction method including the same, according to an embodiment of the invention.

Referring to FIG. 19, the method for learning a 3D geometric structure of a molecule and the target property prediction method including the same, according to an embodiment of the invention, may include: a step (S301) of performing first pre-training based on a 3D conformer encoder (f_3D); a step (S303) of performing second pre-training based on the first pre-trained 3D conformer encoder (f_3D) and a 2D graph encoder (f_2D); a step (S305) of performing third pre-training based on the second pre-trained 2D graph encoder (f_2D); and a step (S307) of providing the third pre-trained 2D graph encoder (f_2D).

In detail, the computing system 1000 according to an embodiment of the invention may perform first pre-training based on a 3D conformer encoder (f_3D). (S301)

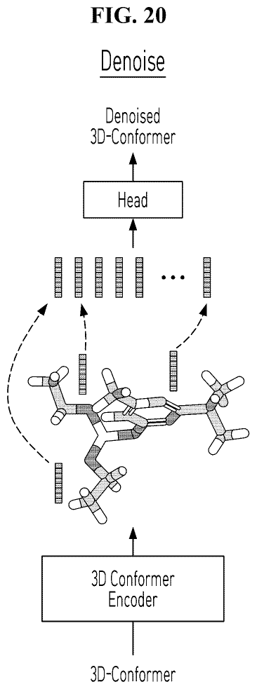

FIG. 20 illustrates an example conceptual diagram for explaining first pre-training according to an embodiment of the invention.

In detail, referring to FIG. 20, in the embodiment, the computing system 1000 may perform denoising-based training (hereinafter, first pre-training) on the 3D conformer encoder (f_3D) using a predetermined 3D molecular dataset as training data.

In more detail, in the embodiment, the computing system 1000 may insert a predetermined noise into each 3D molecular data.

Concretely, in the embodiment, the computing system 1000 may represent each 3D molecular data in the form of (C=(V, R)).

Here, V denotes a set of atoms, and R denotes a matrix containing the coordinates of each atom within a 3D space.

Moreover, in the embodiment, the computing system 1000 may add a predetermined noise (e.g., Gaussian noise, etc.) to each 3D molecular data.

In this case, the computing system 1000 may represent the 3D molecular data modified by the added noise (i.e., 3D noising data) in the form of (\tilde{C}=(V, \tilde{R})).

Accordingly, in the embodiment, the computing system 1000 may obtain a set of 3D molecular data with a predetermined noise inserted in it (i.e., 3D noising data).

In addition, in the embodiment, the computing system 1000 may perform denoising-based training on the 3D conformer encoder (f_3D) using the set of 3D molecular data (i.e., 3D noising data) with noise inserted in it.

In detail, in the embodiment, the computing system 1000 may input each 3D noising data as training data into the 3D conformer encoder (f_3D).

Accordingly, the 3D conformer encoder (f_3D) may perform 3D molecular structure learning based on a given set of 3D noising data through a process of removing noise from the input 3D noising data (that is, a process of restoring to the original data).

More specifically, in the embodiment, the 3D conformer encoder (f_3D) may perform denoising learning using 3D noising data according to the following [Mathematical Formula 8].

$${h}_{3} D ( {f}_{3} D ( \tilde{C} ) ) = ( {\hat{ϵ}}_{1} , … , {\hat{ϵ}}_{N} ) \qquad [ Mathematical Formula 8 ]$$

That is, in the embodiment, the 3D conformer encoder (f_3D) may perform denoising learning in such a manner as to receive atom coordinates with added noise as input and predict (restore) the original atom coordinates.

In this case, the 3D conformer encoder (f_3D) may predict the 3D vector of each atom by adding a prediction head (h_3D) to the output.

Furthermore, in the embodiment, the 3D conformer encoder (f_3D) may perform learning in such a manner as to minimize the difference between a noise vector predicted through each 3D noising data and an actual noise vector, based on the loss function presented in [Mathematical Formula 9].

$${L}_{denoise} = {E}_{p ( \tilde{C} , C )} [ {‖ {h}_{3} D ( {f}_{3} D ( \tilde{C} ) ) - ( {\hat{ϵ}}_{1} , … , {\hat{ϵ}}_{N} ) ‖}_{2}^{2} ] \qquad [ Mathematical Formula 9 ]$$

Here, p(\tilde{C}, C) in [Mathematical Formula 9] denotes a probability distribution derived from the distribution of a given dataset and a noise sampling procedure.

In this case, as previously described, the 3D conformer encoder (f_3D) according to the embodiment has invariance to rotations and translations in the SE(3) group, and therefore can accurately learn the characteristics of a molecular structure (i.e., quantum mechanical property) regardless of spatial transformations.

Accordingly, in the embodiment, the computing system 1000 may perform denoising training on the 3D conformer encoder (f_3D) based on a 3D noising dataset.

Through this, in the embodiment, the computing system 1000 can construct a 3D conformer encoder (f_3D) that is robust to noise, and the 3D conformer encoder (f_3D) thus constructed can more effectively learn key information related to 3D molecular structures, allowing it to be applied to prediction of various molecular properties.

In addition, the computing system 1000 according to an embodiment of the invention may perform second pre-training based on the first pre-trained 3D conformer encoder (f_3D) and the 2D graph encoder (f_2D). (S303)

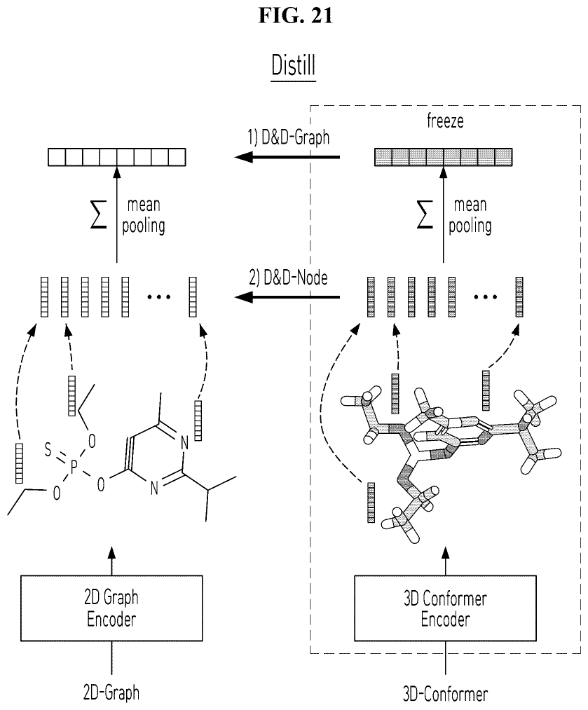

FIG. 21 illustrates an example conceptual diagram for explaining second pre-training according to an embodiment of the invention.

In detail, referring to FIG. 21, in the embodiment, the computing system 1000 may perform second pre-training (i.e., knowledge distillation training) in which knowledge learned by the first pre-trained 3D conformer encoder (hereinafter, 3D conformer denoising encoder) is transferred (distilled) to the 2D graph encoder (f_2D).

Generally, pre-training using the 3D conformer encoder (f_3D) enables learning of generalizable 3D characteristic information through denoising of 3D molecular structures. However, in practical applications, performing high-cost 3D conformer (i.e., 3D molecular data) computations for every molecule is highly inefficient.

Therefore, in the embodiment, the computing system 1000 may perform cross-modal knowledge distillation training to transfer the knowledge learned by the 3D conformer denoising encoder to the 2D graph encoder (f_2D), in order to predict three-dimensional characteristic information with high accuracy using the 2D graph encoder (f_2D).

In more detail, in the embodiment, the computing system 1000 may perform second pre-training in which the 2D graph encoder (f_2D) is trained so that representations outputted by the 2D graph encoder (f_2D) follows (mimics) representations outputted by the 3D conformer denoising encoder.

Specifically, in the embodiment, the computing system 1000 may perform 1) graph-level knowledge distillation (D&D-GRAPH).

In detail, the computing system 1000 may perform graph-level knowledge distillation by minimizing the differences between graph representations outputted by the 2D graph encoder (f_2D) and those outputted by the 3D conformer denoising encoder.

In more detail, in the embodiment, the 3D conformer denoising encoder may receive predetermined 3D molecular data as input and learn corresponding representations for each atom.

Furthermore, the 3D conformer denoising encoder may generate a representation of the entire graph by applying mean pooling to the learned representations.

In the same manner, in the embodiment, the 2D graph encoder (f_2D) may receive predetermined 2D molecular data (G) as input, learn corresponding representations for each atom, and generate a representation of the entire graph by applying mean pooling to the learned representations.

Subsequently, in the embodiment, the computing system 1000 may perform training in such a way as to minimize the L2 loss between a graph representation generated by the 3D conformer denoising encoder (hereinafter, 3D mean-pooled graph representation) and a graph representation generated by the 2D graph encoder (f_2D) (hereinafter, 2D mean-pooled graph representation), based on the loss function presented in [Mathematical Formula 10].

$${L}_{distill - graph} = {‖ pool ( {f}_{2} D ( G ) ) - pool ( {f}_{3} D ( C ) ) ‖}_{2}^{2} \qquad [ Mathematical Formula 10 ]$$

Here, the pool function in [Mathematical Formula 10] refers to a function that generates a graph representation by applying mean pooling to output node representations of each encoder.

In this process, in some embodiments, the computing system 1000 may train and update the 2D graph encoder (f_2D) while keeping the 3D conformer denoising encoder frozen. Further details regarding this will be described later.

Accordingly, in the embodiment, the computing system 1000 may perform graph-level knowledge distillation based on the 3D conformer denoising encoder and the 2D graph encoder (f_2D).

Through this, the computing system 1000 may provide a framework that efficiently reduces data processing costs for predicting 3D characteristic information

In addition, in the embodiment, the computing system 1000 may perform 2) node-level knowledge distillation (D&D-NODE).

In detail, the computing system 1000 may perform node-level knowledge distillation in such a way as to minimize the differences between node representations for each atom outputted by the 2D graph encoder (f_2D) and those outputted by the 3D conformer denoising encoder.

In more detail, in the embodiment, the 3D conformer denoising encoder may receive predetermined 3D molecular data as input and learn corresponding representations for each atom (hereinafter, 3D atom node representations).

In the same manner, in the embodiment, the 2D graph encoder (f_2D) may receive predetermined 2D molecular data as input and learn corresponding representations for each atom (hereinafter, 2D atom node representations).

Subsequently, in the embodiment, the computing system 1000 may perform training in such a way as to minimize the L2 loss between the 3D atom node representations and the 2D atom node representations, based on the loss function presented in [Mathematical Formula 11].

$${L}_{distill - node} = {‖ {f}_{2} D ( G ) - {f}_{3} D ( C ) ‖}_{2}^{2} \qquad [ Mathematical Formula 11 ]$$

In this embodiment, the 3D conformer denoising encoder and the 2D graph encoder (f_2D) each generate a unique representation for each atom, and the computing system 1000 may train the two encoders so that their outputs for each atom match each other.

In this process, in some embodiments, the computing system 1000 may train and update the 2D graph encoder (f_2D) while keeping the 3D conformer denoising encoder frozen. Further details regarding this will be described later.

Accordingly, in the embodiment, the computing system 1000 may perform node-level knowledge distillation based on the 3D conformer denoising encoder and the 2D graph encoder (f_2D).

In this way, the computing system 1000 may construct the 2D graph encoder (f_2D) such that it performs predictions reflecting more detailed knowledge transferred from the 3D conformer denoising encoder, by implementing knowledge transfer of physical and chemical properties to the 2D graph encoder (f_2D) at a finer granularity compared to graph-level knowledge transfer. In the embodiment, as described above, the computing system 1000 may allow the 2D graph encoder (f_2D) to perform high-performance molecular property predictions encompassing three-dimensional characteristic information using 2D molecular data, by implementing knowledge transfer to distill the knowledge learned by the 3D conformer denoising encoder to the 2D graph encoder (f_2D).

On the other hand, in the embodiment, when performing the second pre-training (i.e., knowledge distillation training) as described above, the computing system 1000 may freeze at least some parameters of the 3D conformer denoising encoder.

The goal of the second pre-training is to transfer the knowledge learned by the 3D conformer denoising encoder to the 2D graph encoder (f_2D). To this end, the 3D conformer denoising encoder must remain unchanged (i.e., frozen) during the second pre-training.

Through this, the 3D conformer denoising encoder preserves its previously learned information, while the 2D graph encoder (f_2D) is able to learn its own weights based on that preserved knowledge.

In detail, in the embodiment, the computing system 1000 can freeze at least some parameters of the 3D conformer denoising encoder during the second pre-training process, thereby preventing updates to its weights.

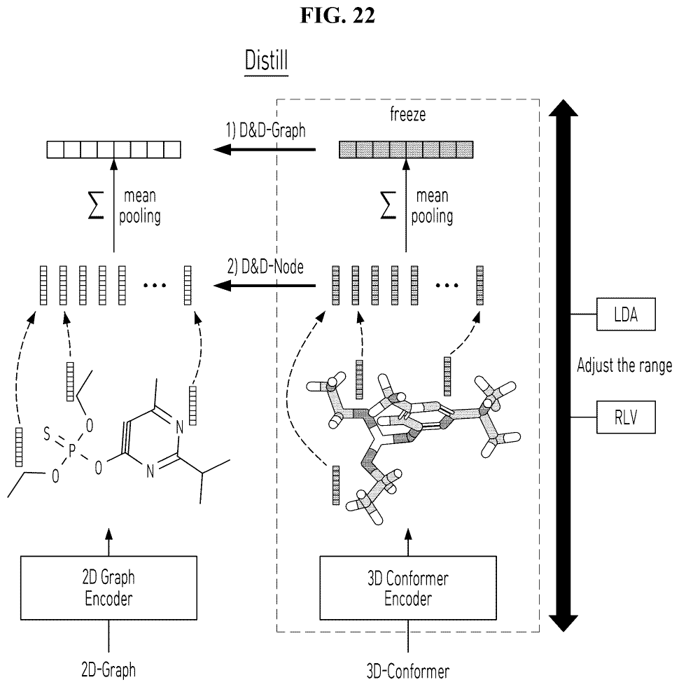

FIG. 22 illustrates an example diagram for explaining a method for determining a model parameter freezing range for second pre-training according to an embodiment of the invention.

In this case, referring to FIG. 22, in some embodiments, the computing system 1000 may determine the freezing range of the 3D conformer denoising encoder based on the amount of training data for second pre-training (LDA; hereinafter, the amount of second pre-training data).

In the embodiment, if the amount of second pre-training data (LDA) is below a predetermined reference value, the computing system 1000 may freeze all parameters of the 3D conformer denoising encoder.

Conversely, if the amount of second pre-training data (LDA) exceeds the predetermined reference value, the computing system 1000 may unfreeze all parameters of the 3D conformer denoising encoder.

In another embodiment, the computing system 1000 may determine the freezing range of the 3D conformer denoising encoder in inverse proportion to the amount of second pre-training data (LDA).

That is, the computing system 1000 may narrow the freezing range of the 3D conformer denoising encoder as the amount of second pre-training data (LDA) increases, and widen the freezing range of the 3D conformer denoising encoder as the amount of second pre-training data (LDA) decreases.

For example, if the amount of second pre-training data (LDA) exceeds a preset first reference value but is equal to or lower than a preset second reference value, the computing system 1000 may freeze n (where n>=1) preset parameters within the 3D conformer denoising encoder. If the amount of second pre-training data (LDA) exceeds the preset second reference value but is equal to or lower than a preset third reference value, the system may freeze n-m (where m>=1) preset parameters within the 3D conformer denoising encoder.

Additionally, referring further to FIG. 22, in some embodiments, the computing system 1000 may determine the freezing range of the 3D conformer denoising encoder based on the relevance (RLV) between the 3D molecular structure and the target property, based on the pre-training data for the second pre-training.

In the embodiment, the computing system 1000 may determine the freezing range of the 3D conformer denoising encoder in inverse proportion to the aforementioned relevance (RLV).

That is, the computing system 100 may narrow the freezing range of the 3D conformer denoising encoder as the relevance (RLV) becomes higher and widen the freezing range of the 3D conformer denoising encoder as the relevance (RLV) becomes lower.

As such, in some embodiments, the computing system 1000 can further enhance the performance of the second pre-training (i.e., knowledge distillation training) by variably determining the freezing range of the 3D conformer denoising encoder in various manners.

Returning to the discussion, the computing system 1000 may also perform the second pre-training while keeping at least a portion of the 3D conformer denoising encoder frozen.

Accordingly, the computing system 1000 can implement the second pre-training in such a way that the parameters of the 2D graph encoder (f_2D) are updated, while the frozen parameters of the 3D conformer denoising encoder remain fixed.

In this manner, in the embodiment, by applying a freezing technique during the second pre-training, the computing system 1000 allows the 3D conformer denoising encoder to preserve its previously learned information, and enables the 2D graph encoder (f_2D) to effectively learn its own weights based on that preserved information.

Additionally, the computing system 1000 according to the embodiment of the invention may perform third pre-training based on the second pre-trained 2D graph encoder (f_2D). (S305)

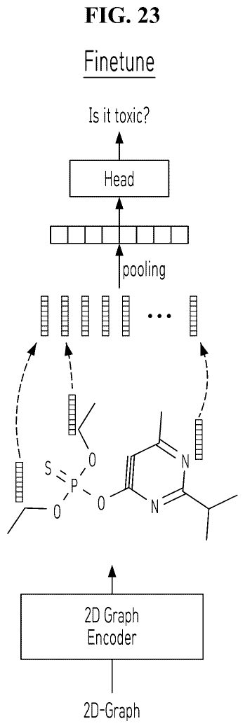

FIG. 23 illustrates an example conceptual diagram for explaining third pre-training according to an embodiment of the invention.

In detail, referring to FIG. 23, in the embodiment, the computing system 1000 may perform third pre-training (i.e., fine-tuning training) to optimize the second pre-trained 2D graph encoder (hereinafter, the 2D graph transfer encoder) for a predetermined target property.

That is, in the embodiment, the computing system 1000 may fine-tune the 2D graph transfer encoder using a dataset for a predetermined target property (e.g., boiling point, melting point, surface tension, and/or solubility), thereby constructing an encoder optimized for each target property.

In detail, in the embodiment, the computing system 1000 may collect a predetermined target property dataset (e.g., an Open Graph Benchmark (OGB) dataset and/or a manually curated physical molecular property dataset).

Here, the aforementioned target property dataset may data that specifies molecular structural information and/or a target property value.

In addition, in the embodiment, the computing system 1000 may perform a downstream task on the 2D graph transfer encoder using the collected target property dataset.

For reference, the downstream task may refer to a process of optimizing (tuning) a pre-trained model for a real-world application problem.

That is, in the embodiment, the computing system 1000 may perform a downstream task to optimize the 2D graph transfer encoder for a specific task such as predicting a predetermined target property.

In the embodiment, the computing system 1000 may perform a downstream task based on full model finetuning in which all parameters of the 2D graph transfer encoder are fine-tuned using a target property dataset.

In such cases, the computing system 1000 can increase the likelihood of deriving the best local optimum for the target property, thereby enhancing both prediction performance and reliability.

In another embodiment, the computing system 1000 may perform a downstream task based on prediction head finetuning in which only the final prediction head of the 2D graph transfer encoder is fine-tuned using the target property dataset.

That is, the computing system 1000 may perform a downstream task by fine-tuning only the final prediction head (i.e., the parameters for outputting the final predicted value) while keeping the remaining layers frozen.

In such cases, the computing system 1000 can increase training speed and minimize overfitting.

In this case, in the embodiment, the computing system 1000 may perform the above third pre-training using various disclosed optimization algorithms (e.g., AdamW).

In this way, the computing system 1000 can fine-tune the 2D graph transfer encoder for a predetermined target property, thereby constructing the 2D graph transfer encoder in a manner that internalizes knowledge optimized for that specific property.

Through this, the computing system 1000 can support the 2D graph transfer encoder in performing accurate and generalized predictions that align with particular application cases.

Additionally, the computing system 1000 can accordingly enable the 2D graph transfer encoder to output high-quality predicted values for a target property, even in environments with limited labeled data.

Furthermore, the computing system 1000 according to an embodiment of the invention may provide the third pre-trained 2D graph encoder (f_2D). (S307)

In detail, in the embodiment, the computing system 1000 may provide the third pre-trained 2D graph encoder (hereinafter, 2D graph fine-tuning encoder) in a predetermined manner.

In the embodiment, the computing system 1000 may apply and provide the 2D graph fine-tuning encoder to a predetermined multitasking model.

For example, the computing system 1000 may provide the above-described 2D graph fine-tuning encoder by replacing the encoder module (ECM) of the above multitasking learning model (MtLM) with the 2D graph fine-tuning encoder.

Additionally, in some embodiments, the computing system 1000 may provide the above trained 2D graph fine-tuning encoder by applying them to actual application services.

Specifically, the encoder may be deployed as the core engine of a multitasking model that either simultaneously predicts a plurality of properties (characteristics) or generates a new molecule that satisfy a desired combination of properties.

However, when a user utilizes such a multitasking model, there may be cases where they input target property values for a domain that is physically infeasible or is not sufficiently learned by the model. These inappropriate input values become a primary cause of molecular generation failure.

Therefore, to address this issue and enhance both the usability and reliability of the model, the computing system 1000 may provide the encoder in the form of a “guided service” that assists in improving the performance of the model equipped with the encoder.

In this case, the aforementioned guided service may serve to pre-evaluate how appropriate the user's input values are for molecular generation, and provide guidance that steers the user input toward a direction that increases the likelihood of success.

Specifically, the computing system 1000 may first acquire first input information from the user that includes a plurality of target property values that a desired molecule is expected to satisfy.

Additionally, the computing system 1000 may acquire a feasibility indicator, which quantitatively specifies the difficulty of generating output data (e.g., molecular structural formula) from the multitasking model based on the acquired first input information.

Here, the feasibility indicator is calculated such that the lower the difficulty of generation (i.e., the higher the likelihood of success), the higher its value. The feasibility indicator may be acquired by at least one of the following steps: estimating a density value within a training data distribution based on a predetermined density estimation algorithm, detecting outliers based on a predetermined anomaly detection algorithm, and/or calculating similarity to actual data based on a predetermined similarity assessment algorithm.

Furthermore, if the feasibility indicator acquired as above is below a preset reference value, the computing system 1000 may generate and provide to the user first guidance information (property guidance) which suggests making changes to the target properties in a direction that decreases the difficulty of generation, or second guidance information (learning guidance) which suggests supplementing the training data to improve the model's performance.

As such, in the embodiment, the computing system 1000 may provide a D&D (Denoise and Distill) framework that enables the 2D graph encoder (f_2D) (i.e., the 2D graph fine-tuning encoder) to perform high-accuracy and high-quality molecular property predictions even in the absence of explicit 3D molecular structure information.

Accordingly, in the embodiment, the computing system 1000 can directly enhance the overall quality and performance of related industries and services.

The method for learning a 3D geometric structure of a molecule and the target property prediction method including the same, according to an embodiment of the invention, can pre-train a 2D data-based encoder based on a D&D (Denoise and Distill) framework using 3D data, thereby enabling the 2D-level encoder to efficiently incorporate 3D-level information.

Accordingly, the method for learning a 3D geometric structure of a molecule and the target property prediction method including the same, according to an embodiment of the invention, can provide a high-performance encoder that performs molecular property predictions based on 3D-level information by using two-dimensional data (e.g., 2D molecular graphs, etc.).

Thus, the method for learning a 3D geometric structure of a molecule and the target property prediction method including the same, according to an embodiment of the invention, can achieve high prediction accuracy by utilizing 3D-level information, while significantly reducing computational cost.

Moreover, the method for learning a 3D geometric structure of a molecule and the target property prediction method including the same, according to an embodiment of the invention, can perform denoising-based training through a 3D conformer encoder (f_3D) and then transfer (distill) the knowledge learned by the 3D conformer encoder (f_3D) to a 2D graph encoder (f_2D), thereby providing a high-performance encoder that combines the strengths of 3D-based molecular representation learning with the efficiency of 2D-based molecular representation learning.

In addition, the method for learning a 3D geometric structure of a molecule and the target property prediction method including the same, according to an embodiment of the invention, can accordingly implement a 2D-level encoder that effectively learns generalized knowledge from a given dataset, thereby maintaining high prediction performance even in environments with limited labeled data.

Furthermore, the method for learning a 3D geometric structure of a molecule and the target property prediction method including the same, according to an embodiment of the invention, can perform learning in a manner that optimizes (fine-tunes) the knowledge-distilled 2D graph encoder (f_2D) for a specific property, thereby enabling accurate and generalized predictions that align with particular application cases.

Furthermore, the method for learning a 3D geometric structure of a molecule and the target property prediction method including the same, according to an embodiment of the invention, can enhance the efficiency of learning and prediction of large-scale data while simultaneously improving the versatility and scalability of such learning and prediction, by providing the above-described D&D (Denoise and Distill) framework.

That is, the method for learning a 3D geometric structure of a molecule and the target property prediction method including the same, according to an embodiment of the invention, provide a generalized learning framework that is not limited to specific molecular property prediction problems but can be applied to a variety of molecular science problems, thereby providing an improved encoder that can be broadly utilized across various research and industrial applications.

The embodiments of the invention described above may be implemented in the form of program instructions that can be executed through various computer components and recorded on a computer-readable medium. The computer readable medium may include program instructions, data files, data structures, and the like alone or in combination. The program instructions recorded on the computer-readable recording medium may be specially designed and configured for the invention, or may be known and available to those skilled in computer software. Examples of the computer-readable recording medium include: magnetic media such as hard disks, floppy disks, and magnetic tape; optical media such as CD-ROMs and DVDs; magneto-optical media such as floptical disks; and hardware devices such as ROM, RAM, and flash memory specifically configured to store and execute program instructions. Examples of the program instructions include machine language codes such as those generated by a compiler, as well as high-level language codes that can be executed by a computer using an interpreter or the like. The hardware devices may be configured to act as one or more software modules in order to perform processing according to the invention, and vice versa.

The specific implementations described in the invention are exemplary embodiments, and do not limit the scope of the invention in any way. For brevity of the specification, descriptions of conventional electronic configurations, control systems, software, and other functional aspects of the systems may be omitted. In addition, the connections or connection members of lines between the components shown in the drawings are illustrative examples of functional connections and/or physical or circuit connections, and in actual devices, may be shown as alternative or additional various functional connections, physical connections, or circuit connections. In addition, unless specifically mentioned, such as “essential”, “importantly”, etc., the components described herein may not be necessary components for application of the invention.

The invention relates to a method of training a multitasking model and a method for performing multitasking using a machine learning model trained based on this method, which have industrial applicability since they are applicable to the artificial intelligence industry.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A computer-implemented method for learning a 3D geometric structure of a molecule and predicting a target property, comprising:
performing denoising-based, first pre-training based on a 3D conformer encoder configured to input 3D molecular data specifying a 3D-level molecular structure;
performing knowledge distillation-based, second pre-training based on the first pre-trained 3D conformer encoder and a 2D graph encoder configured to input 2D molecular data specifying a 2D-level molecular structure;
performing fine-tuning based, third pre-training based on the second pre-trained 2D graph encoder; and
providing the third pre-trained 2D graph encoder.

2. The method of claim 1, wherein the performing of first pre-training comprises:
inserting a predetermined noise into the 3D molecular data; and
training the 3D conformer encoder to restore the 3D molecular data with the inserted noise to the original 3D molecular data and learn the 3D-level molecular structure.

3. The method of claim 2, wherein the performing of first pre-training further comprises learning data representations invariant to rotations and translations in a 3D space, based on a predetermined SE(3) permutation invariant architecture.

4. The method of claim 1, wherein the performing of second pre-training comprises performing distillation training in which a 3D conformer denoising encoder, which is the first pre-trained 3D conformer encoder, serves as a teacher model, and the 2D graph encoder serves as a student model.

5. The method of claim 4, wherein the performing of distillation training comprises training the 2D graph encoder so that representations outputted by the 2D graph encoder follow representations outputted by the 3D conformer denoising encoder.

6. The method of claim 5, wherein the performing of distillation training further comprises performing graph-level knowledge distillation (D&D-GRAPH) to minimize the differences between graph-level representations outputted by the 2D graph encoder and graph-level representations outputted by the 3D conformer denoising encoder.

7. The method of claim 5, wherein the performing of distillation training further comprises performing node-level knowledge distillation (D&D-NODE) to minimize the differences between node-level representations outputted by the 2D graph encoder and node-level representations outputted by the 3D conformer denoising encoder.

8. The method of claim 5, wherein the performing of distillation training further comprises freezing at least some parameters of the 3D conformer denoising encoder.

9. The method of claim 1, wherein the performing of third pre-training further comprises performing a downstream task to optimize a 2D graph transfer encoder which is the second pre-trained 2D graph encoder.

10. The method of claim 1, wherein the providing of the third pre-trained 2D graph encoder comprises applying a 2D graph fine-tuning encoder, which is the third pre-trained 2D graph encoder, to a predetermined multitasking model.

11. The method of claim 1, wherein:
the providing of the third pre-trained 2D graph encoder comprises:
evaluating the feasibility of first input information specifying a plurality of target properties inputted by a user, through a multitasking model including the encoder; and
providing guidance based on a result of the evaluation; and
the providing of guidance comprises:
acquiring a feasibility indicator quantitatively specifying the difficulty of generation of output data from the multitasking model based on the first input information; and
if the acquired feasibility indicator is below a preset reference value, generating and outputting guidance information for decreasing the difficulty of generation.

12. The method of claim 11, wherein the acquiring of a feasibility indicator comprises calculating the feasibility indicator for the first input information based on at least one of a predetermined density estimation algorithm, a predetermined anomaly detection algorithm, or a predetermined similarity assessment algorithm.

13. The method of claim 11, wherein the guidance information comprises at least one of first guidance information which suggests making changes to the target properties to decrease the difficulty of generation, or second guidance information which suggests supplementing the training data to improve the model's performance.

14. A system for learning a 3D geometric structure of a molecule and predicting a target property, the system comprising:
at least one memory; and
at least one processor configured to retrieve at least one application stored in the memory to learn a 3D geometric structure of a molecule and predict a target property,
wherein instructions of the processor include instructions for executing the steps of:
performing denoising-based, first pre-training based on a 3D conformer encoder configured to input 3D molecular data specifying a 3D-level molecular structure;
performing knowledge distillation-based, second pre-training based on the first pre-trained 3D conformer encoder and a 2D graph encoder configured to input 2D molecular data specifying a 2D-level molecular structure;
performing fine-tuning-based, third pre-training based on the second pre-trained 2D graph encoder; and
providing the third pre-trained 2D graph encoder.
